(19) United States
(12) Patent Application Publication
(10) Pub. No.: US 2006/0240494 A1
(43) Pub. Date: Oct. 26, 2006

(54) Biocidal molecules, macromolecular targets and methods of production and use

(75) Inventors: Laszlo Otvos, Audubon, PA (US); Magdalena Blaszczyk-Thurin, Philadelphia, PA (US); Mark Rogers, Glenmoore, PA (US); Sandor Lovas, Omaha, NE (US)

Correspondence Address: HOWSON AND HOWSON, SUITE 210, 501 OFFICE CENTER DRIVE, FT WASHINGTON, PA, 19034, US

(73) Assignees: The Wistar Institute of Anatomy and Biology, Philadelphia, PA (US); Creighton University, Omaha, NE (US)

(21) Appl. No.: 11/477,089

(22) Filed: Jun. 28, 2006

Related U.S. Application Data
(63) Continuation of application No. 10/181,654, filed on Sep. 27, 2002.
(60) Provisional application No. 60/237,599, filed on Oct. 3, 2000.
(60) Provisional application No. 60/177,565, filed on Jan. 21, 2000.

(51) Int. Cl. G01N 33/554; C12Q 1/18; A61K 38/16; A01N 65/00

(52) U.S. Cl. 435/7.32; 435/32; 514/12

(57) ABSTRACT

A method for identifying a compound that has a biocidal effect against a selected organism involves screening from among known or unknown peptide or non-peptide molecules, a test molecule that binds selectively to a target sequence of a multi-helical lid of a heat shock protein of the organism. The binding of the test compound inhibits the protein folding activity of the protein. A specific embodiment of such a method is useful for identifying or designing a pharmaceutical or veterinary biocidal or antibiotic compound, preferably a pathogen and/or strain-specific compound. For this purpose, the compound does not bind to a heat shock protein that is homologous to the mammalian subject to be treated with the compound. Screening methods can encompass direct binding or competitive assays. Molecules or compounds identified by these methods are employed as biocides for pharmaceutical, veterinary, pesticide, insecticide and rodenticide uses, among others.

### CROSS-REFERENCE TO RELATED APPLICATIONS

This is a continuation of U.S. patent application Ser. No. 10/181,654, which entered the National Stage under 35 U.S.C. §371 on Sep. 27, 2002 of PCT/US01/01812, filed Jan. 19, 2001, which claims the benefit under 35 USC 119(e) of prior U.S. Provisional Patent Application Nos. 60/237,599, filed Oct. 3, 2000, and 60/177,565, filed Jan. 21, 2000.

### STATEMENT REGARDING FEDERALLY SPONSORED RESEARCH OR DEVELOPMENT

This invention was supported in part by National Institute of Health Grant No. GM45011 and National Science Foundation Grant No. EPS-9720643. The United States government has an interest in this invention.

### BACKGROUND OF THE INVENTION

The invention relates generally to methods for identifying and screening biocidal compositions, e.g., such as compositions useful for treating pathogenic infections in mammals. More specifically, the methods and compositions described herein employ the interaction between a modified, or synthetic peptide and a targeted receptor present on a heat shock protein of the pathogen.

The incidence of serious antibacterial infection is increasing despite remarkable advances in antibiotic chemotherapy. Each year there are more than 40 million hospitalizations in the United States. About 2 million hospital patients acquire nosocomial infections, 50 to 60 percent of which involve antibiotic-resistant bacteria; the number of deaths related to nosocomial disease is estimated at 60,000-70,000 annually [Thomasz, A. (1994) New Engl. J Med., 330: 1247-1251]. The past decade has seen a climb in number of incidents with multi-drug resistant Gram-positive strains [Moellering, R. C, Jr. (1998) Clin. Infect. Dis., 26: 1177-1178]. Methicillin-resistant Staphylococcus aureus is now emerging in distinctly different community-acquired strains that are susceptible to more antibiotics, but may be more efficiently transmitted than their nosocomial counterparts.

In the past, the solution to bacterial resistance has been primarily dependent on the development of clinically viable anti-microbial agents [Adcock, P. M. et al, (1998) J. Infect. Dis., 78: 577-580; Maguire, G. P. et al, (1998) J. Hosp. Infect., 38: 273-281]. One of the most serious needs of the health-care industry today is the rapid development of antibacterial compounds that kill bacteria in a manner completely different from those utilized by the currently marketed antimicrobial compounds, such as erythromycin, tetracyclines, penicillins, cephalosporins and even vancomycin.

Apart from the discovery of natural antibacterial peptides from plants and animals, there have been few new antibiotics developed in recent years [Tan, Y.-T., Tillett, D. J., and McKay, I. A. (2000) Mol. Med. Today 6:309-314]. In addition, it is now widely accepted that the traditional screening methods, based on direct measurements in living cells of the inhibitory capacities of particular compounds, are unlikely to generate many promising molecules [Giglione, C. et al, (2000) Mol. Microbiol., 36: 1197-1205]. The validated conditions pharmaceutical companies prefer often fail to reproduce the results obtained at research laboratories, probably because the validated assay is concerned with the reproduction of bacteria in specific media and conditions most suitable for bacterial growth, conditions not present in vivo in mammals.

Most antimicrobial peptides kill bacteria by inhibiting some bacterial functions, but do not have a specific macromolecular target. Some peptides kill bacteria by disrupting the cell membrane or cell wall. For example, the cecropins, defensins and magainins all act on the cell membrane [Otvos, L., Jr. (2000) J. Pept. Sci., 6: 497-511]; buforin II binds non-specifically to bacterial DNA [Park, C. B. et al, (1998) Biochem. Biophys. Res. Commun., 244:253-257]. Some other antimicrobial peptides, such as the histatins or NAP-2, are known to act as inhibitors of enzymes produced by the bacteria either by serving as a pseudo-substrate or by tight binding to the active site eliminating the accessibility of the native substrate [Andreu, D., and Rivas, L. (1998) Biopolymers, 47: 415433].

Perhaps the most promising among the antibacterial peptides are the insect-derived, small, proline-rich, antibacterial peptides that bind to an unknown, stereospecific target molecule [P. Bulet et al, (1996) Eur. J Biochem 238:64-69; Kabsch, W., and Sander, C. (1983) Biopolymers, 22:2577-2637; D. Hultmark, (1993) Trends Genet., 9:178-183; J. P. Gillespie et al, (1997) Ann. Rev. Entomol., 42:611-643]. See, also, International Patent Publication No. WO94/05787, published Mar. 17, 1999; International Patent Publication No. WO99/05270, published Feb. 4, 1999; and International Patent Publication No. WO97/30082, published Aug. 21, 1997. Two such peptides are drosocin, a 19 amino acid residue peptide from species of Drosophila [P. Bulet et al, (1993) J. Biol. Chem., 268(20):14893-14897] and pyrrhocoricin, a 20 amino acid residue peptide from species of Pyrrhocoris [S. Cociancich et al, (1994) Biochem. J., 300:567-575]. Drosocin and pyrrhocoricin are glycopeptides characterized by the presence of a disaccharide in the mid-chain position. The presence of the sugar increases the in vitro antibacterial activity of drosocin, but decreases the activity of pyrrhocoricin [P. Bulet et al, 1996, cited above; R. Hoffmann et al, (1999) Biochim et Biophys. Acta, 1426:459-467]. Both drosocin and pyrrhocoricin are tentatively assigned to the proline-rich peptide family that includes other members, such as apidaecin, abaecin, metchnikowin and lebocin [Gillespie, J. P. et al, (1997) Annu. Rev. Entomol., 42: 611-643].

Drosocin is moderately active against Gram-positive bacteria. When the native glycosylated drosocin is injected into mice, the glycopeptide shows no antibacterial activity, probably due to the peptide's rapid decomposition in mammalian sera [Hoffmann et al, 1999, cited above]. While drosocin needs 24 hours to kill bacteria in vitro, it is completely degraded in diluted human and mouse serum within a four-hour period. Both aminopeptidase and carboxypeptidase cleavage pathways (decomposition at both ends) can be observed.

Native pyrrhocoricin is also a glycosylated peptide. Pyrrhocoricin is more active against Gram-negative bacteria than drosocin, but the peptide is almost completely inactive against Gram-positive strains. Native pyrrhocoricin appears to be more resistant to mouse serum degradation than drosocin, but decomposes quickly in some batches of human serum. Pyrrhocoricin is significantly more stable, has increased in vitro efficacy against Gram-negative bacterial strains, and is devoid of in vitro or in vivo toxicity. At low doses, pyrrhocoricin protected mice against E. coli infection, but at a higher dose was toxic to compromised animals [Otvos et al, (2000) Protein Science, 9:742-749].

Metabolites from serum stability assays of drosocin and pyrrhocoricin were identified, and metabolites lacking as few as five amino terminal or two carboxy terminal amino acids were inactive [Bulet et al, 1996 and Hoffmann et al, 1999, both cited above]. This observation was further supported by a recent model of the bioactive secondary structure of drosocin, which identifies two reverse turns, one at each terminal region, as binding sites to the target molecule [A. M. McManus et al, (1999) Biochem., 38(2):705-714]. The situation is further complicated by the fact that the degradation speed and pathway of a given peptide in diluted mouse sera are somewhat different from those observed in diluted human sera. Even different batches of human sera degrade the peptides at different rates and may yield different metabolites in vitro. The peptide's stability is markedly increased in insect hemolymph where the peptides manifest their biological functions [Hoffmann et al, (1999), cited above].

Drosocin and pyrrhocoricin share a great deal of sequence homology with other insect antibacterial peptides. A comparison of portions of the sequences of several of such peptides is illustrated in Table 1.

TABLE 1

| | | | |
|---|---|---|---|
| | | | SEQ ID |
| Protein Name | Origin | Sequence^{1,2} | NO: |

| | | | |
|---|---|---|---|
| drosocin | Drosophila | --Gly-Lys-Pro-Arg-Pro-Tyr-Ser-Pro- | 1 |
| | melanogaster | Arg-Pro-Thr-Ser-His-Pro-Arg-Pro-Ile- | |
| | | Arg-Val-- | |
| formaecin 1 | Myrmecia | --Gly-Arg-Pro-Asn-Pro-Val-Asn-Asn- | 2 |
| | gulosa | Lys-Pro-Thr-Pro-Tyr-Pro-His-Leu-- | |
| pyrrhocoricin | P. apterus | --Val-Asp-Lys-Gly-Ser-Tyr-Leu-Pro- | 3 |
| | | Arg-Pro-Thr-Pro-Pro-Arg-Pro-Ile- | |
| | | Tyr-Asn-Arg-Asn-- | |
| apideacin 1a | Apis mellifera | Gly-Asn-Asn-Arg-Pro-Val-Tyr-Ile- | 4 |
| | | Pro-Gln-Pro-Arg-Pro-Pro-His-Pro- | |
| | | Arg-Ile-- | |
| diptericin | Phormia | Asp-Glu-Lys-Pro-Lys-Leu-Ile-Leu- | 5 |
| | terranovae | Pro-Thr-Pro-Ala-Pro-Pro-Asn-Leu-Pro- | |
| | | Gln- | |

¹Glycosylated threonines are underlined.
²Common amino acids are in bold.

Apidaecin, drosocin and pyrrhocoricin were suggested to kill bacteria by acting stereospecifically on a bacterial protein [Bulet, P. et al, (1996) Eur. J. Biochem., 238: 64-69; Casteels, P., and Tempst, P. (1994) Biochem. Biophys. Res. Commun., 199: 339-345; Hoffmann, R. et al, (1999) Biochim. Biophys. Acta, 1426: 459-467]. The proposed mechanism by which apidaecin kills bacteria involves an initial, nonspecific encounter of peptide with an outer membrane component. Thereafter, invasion of the periplasmic space occurs. Invasion is mediated by a specific and essentially irreversible engagement with a receptor/docking molecule that may be inner membrane-bound or otherwise associated. Most likely, the docking molecule is a component of a permease-type transporter system. In the final step, the peptide is translocated into the interior of the cell where it meets its ultimate target, perhaps one or more components of the protein synthesis machinery [Castle, M et al, (1999) J. Biol. Chem., 274, 32555-32564].

There exists a need in the art for novel pathogen and strain-specific, biocidal compounds, novel pharmaceutical or veterinary compositions employing such compounds, and methods of use thereof, as well as novel compounds that can be employed in drug screening analyses to detect and develop new pharmaceutical or veterinary biocidal compositions. There exists a need for assays and assay methods, the readout of which is more representative for the mode of action of the particular biocidal molecule, and the in vivo conditions.

### SUMMARY OF THE INVENTION

In one aspect, the invention provides a method for identifying a compound that has a biocidal effect against a selected organism. This method comprises screening from among known or unknown molecules (e.g., proteinaceous or non-proteinaceous, naturally-occurring or synthetic), a test molecule that binds selectively to a target sequence of a multi-helical lid of a heat shock protein of the selected organism. The protein comprises multiple hinge regions flanked by adjacent helices. Generally the binding inhibits the protein folding activity of the protein, and more specifically, the binding physically restrains essential movement of at least one hinge region. This method is useful for developing compositions directed against a variety of organisms, including bacteria, fungi, parasites, mycobacteria, insects, and non-human ‘pest’ animals, e.g., rodents. Useful target sequences include peptides having homology to the three dimensional structure of the E. coli DnaK protein D-E helix domainsequence IEAKMQELAQVSQKLMEIAQQQHAQQQTA GADA [SEQ ID NO: 6] or to smaller fragments thereof. With each species target sequence are included sequences having at least 65% amino acid homology to the identified D-E helix target sequence.

In another aspect, the invention provides a method for designing a compound that has a biocidal effect against a selected organism. This method involves modifying or synthesizing a molecule to bind selectively to, and physically restrain the essential movement of, a target sequence of a heat shock protein of the selected organism. The binding thus inhibits the protein folding activity of the protein. In certain cases, it is preferable that the molecule does not bind to, or immobilize, a homologous heat shock protein of mammalian, particularly primate, origin. In one embodiment, the molecule anchors two adjacent helices of the protein by ionic bridges between the molecule and each helix. The anchored molecule constrains normal movement in the hinge region.

In still another aspect, the invention provides a method for identifying or designing an antibacterial pharmaceutical or veterinary compound comprising screening from among known or unknown compounds for a test compound that binds selectively to a target sequence of a bacterial heat shock protein. Preferably, the test compound does not bind to a homologous heat shock protein of mammalian origin. The method identifies antibacterial compounds effective against bacteria, e.g., bacteria from the genera Escherichia, Streptococcus, Staphylococcus, Enterococcus, Pseudomonas, Haemophilus, Moraxella, Neisseria, Helicobacter, Aerobacter, Borellia, and Gonorrheae.

In one specific embodiment, this method comprises the steps of employing, in a computer-modeling program, a heat shock protein of a selected non-human organism; generating a high resolution, three-dimensional structure of the heat shock protein; and designing or selecting a peptide or non-peptide compound that binds to the protein and does not bind to a homologous mammalian heat shock protein.

In yet another aspect, the invention provides a method of designing a biocidal composition comprising steps including providing a three-dimensional structure of a heat shock protein of a target non-human organism, the protein having multiple helices, with hinge regions defined by two of the helices. The method includes the step of generating a molecule to specifically bind at least one of the hinge regions of the heat shock protein and then assaying the molecule for its ability to restrict the movement of one or more of the hinge regions. In one embodiment, this method may be computer-implemented.

In still another related aspect, the invention provides a computer program that implements the methods disclosed herein.

In still another aspect, the invention provides a method for identifying an antibacterial pharmaceutical or veterinary compound, the method comprising the steps of performing a competitive assay with (i) a pathogen having a heat shock protein; (ii) a peptide of the pyrrhocoricin-apidaecin-drosocin family of peptides, an analog or derivative thereof, and (iii) a test compound or molecule; and identifying the test compound that competitively displaces the peptide of the pyrrhocoricin-apidaecin-drosocin family of peptides, an analog or derivative thereof from binding to the heat shock protein.

In another aspect, the invention provides a composition comprising a molecule that binds to a selected multi-helical lid of a heat shock protein of a selected organism, wherein the molecule inhibits the protein folding activity of the heat shock protein; and a suitable carrier. Exposure of the organism to this composition retards the growth and reproduction thereof. Thus, such compositions may include pharmaceutical or vaccine compositions for administration to mammals, especially humans, plant pesticides, insecticides, fungicides, and rodenticides, among others. In one embodiment, a useful peptide molecule comprises modified peptides based on the amino acid sequence of pyrrhocoricin, VDKGSYLPRPTPPRPIYNRN [SEQ ID NO: 3].

In still a further aspect, the invention provides a method of treating a mammal for a pathogenic infection comprising administering to a mammalian subject with the infection an effective amount of a molecule that binds selectively to a target sequence of a bacterial heat shock protein. Preferably the molecule does not bind to a homologous heat shock protein of mammalian origin. Such molecules are identified in the context of the compositions described herein.

In another aspect, the invention provides a method of eliminating a plant, insect or animal pest comprising administering to a site of the pest infestation a composition as described above.

In yet a further aspect, the invention provides a peptide fragment of a non-human organism's heat shock protein or target sequence thereof that acts as a receptor for a ligand that does not bind a homologous mammalian, particularly a primate, heat shock protein. Preferably, the bacterial heat shock protein is DnaK and the mammalian heat shock protein is human Hsp60 or Hsp70.

In still another aspect, the invention provides an isolated peptide fragment of a bacterial heat shock protein for use in a screening assay for a biocidal compound or molecule, the fragment having homology to the three dimensional structure of the E. coli DnaK protein D-E helix sequence IEAKMQELAQVSQKLMEIAQ QQHAQQQTAGADA [SEQ ID NO: 6] or to smaller fragments thereof. Within each species of organism, sequences having at least 65% amino acid homology to the specified D-E helix target sequence are also themselves target sequences.

In another aspect, the invention provides a method for treating a bacterial infection comprising administering to a mammalian subject with the infection an effective amount of a molecule that binds selectively to a target sequence of a bacterial heat shock protein, but does not bind to a homologous heat shock protein of mammalian origin.

In a further aspect, the invention provides a molecule that penetrates the peptidoglycan layer of a bacterial cell wall, comprising a transport peptide covalently linked to a second compound that has a biological activity within the cell. The transport peptide may be a member of the pyrrhocoricin-apidaecin-drosocin family or a derivative or analog thereof. Methods for studying a bacterial cell may employ this molecule. Also provided is a method of preparing a pharmaceutical or veterinary compound useful for the treatment of a bacterial infection in a human or animal by transporting a desired compound across the cell wall of Gram-negative bacteria. The method involves covalently linking the desired compound to the above-described transport peptide. A related aspect includes the composition itself, which contains in a physiologically acceptable carrier, a molecule that penetrates the peptidoglycan layer of a bacterial cell wall. In a further aspect, the invention provides a method of treating a patient with a bacterial infection comprising administering to the patient an effective amount of the compound described above.

In yet another aspect, the invention provides a compound identified by the above-defined methods.

Other aspects and advantages of the present invention are described further in the following detailed description of the preferred embodiments thereof.

### BRIEF DESCRIPTION OF THE DRAWINGS

FIG. 1A is a bar graph showing the inhibition of ATPase activity of recombinant E. coli DnaK by synthetic antibacterial peptides, L-pyrrhocoricin, SEQ ID NO: 3 with all amino acids in the L configuration (L-Pyrr), D-pyrrhocoricin, SEQ ID NO: 3 with all amino acids in the D configuration (D-Pyrr), Cecropin A, Magainin II and Drosocin, in an EnzChek ATPase assay.

FIG. 1B is a bar graph showing the inhibition of ATPase activity of recombinant E. coli DnaK by two synthetic pyrrhocoricin fragments, Pyrr_{AA1-9} [aa 1-9 of SEQ ID NO: 3] and Pyrr_{AA10-20} [aa10-20 of SEQ ID NO: 3], as well as the full length pyrrhocoricin peptide [SEQ ID NO: 3] in an EnzChek ATPase assay.

FIG. 2A is a bar graph showing the inhibition of β-galactosidase activities of live E. coli TG-1 cells by synthetic antibacterial peptides, L-Pyrr, D-Pyrr, Drosocin, Buforin II, Magainin II and Conantokin G (ConG).

FIG. 2B is a bar graph showing the inhibition of alkaline phosphatase activities of live E. coli TG-1 cells by the same synthetic antibacterial peptides used in FIG. 2A.

FIG. 3A is a bar graph showing the fluorescence polarization analysis of binding of synthetic DnaK fragments E. coli aa513-551 (referred to as EcA-B) [aa513-551 of SEQ ID NO: 10], E. coli aa583-615 (referred to as EcD-E) [aa583-615 of SEQ ID NO: 10] and S. aureus aa554-585 (referred to as SaD-E) [SEQ ID NO:34] to labeled Pyrrhocoricin (Pyrr), Drosocin (Dros), and Apidaecin (Api). The slashes separate the DnaK helix regions and the labeled antibacterial peptides. The term ‘neg’ stands for the negative control fluorescein-labeled peptide NTDGSTDYGILQINSR [SEQ ID NO:8]. The horizontal lines crossing the bars represent the polarization value of the individual labeled peptides at 1 nM concentration, without addition of any DnaK fragment. These background readings were recorded immediately before and/or after the test peptides were assayed.

FIG. 3B is a dose-response-curve of the E. coli D-E helix hinge peptide [aa583-615 of SEQ ID NO: 10] against N-terminally fluorescein-labeled pyrrhocoricin. For these measurements 10 consecutive readings were averaged. Both experiments representing the two panels were repeated with freshly lyophilized samples and yielded very similar results.

### DETAILED DESCRIPTION OF THE INVENTION

Using a combination of immunoaffinity purification, mass spectrometry and a series of biochemical assays, the inventors have determined that the elusive target proteins to which certain antibacterial proteins pyrrhocoricin, apidaecin and drosocin bind are heat shock proteins. Preferably the heat shock proteins are members of the 70 kDa family of heat shock/chaperone proteins. Based on this discovery, the present invention supplies the need in the art for methods for identifying and designing species-specific biocidal molecules directed against mammalian pathogens including bacteria, mycobacteria, parasites, and fungi, and against certain disease vectors and agricultural pests, such as plant pathogens, insects and rodents. The methods and compositions of this invention are thus useful in the pharmaceutical and veterinary fields and in the agricultural fields based on the binding of the designed or identified molecule to a species-specific heat shock/chaperone protein. Moreover, this invention enables the identification of at least one specific target fragment of a bacterial heat shock protein, and homologous fragments of other species heat shock proteins that act as selective receptors for biocidal molecules. Thus, organism- and strain-specific molecules can be designed for the above uses.

I. Identification of the Heat Shock Protein As Target and Receptor for Biocidal Molecules

The biocidal receptor identified by the inventors that forms the basis of the methods of this invention is the 70 kDa heat shock protein family (Hsp70). The Hsp70 proteins, which can be found in almost all organisms and cell types, are indispensable components of well-functioning cells. The Hsp70 proteins are a class of molecular chaperones, which are required for the proper folding of most in vivo proteins. These molecular chaperons bind to nascent polypeptide chains on ribosomes, and assist in preventing premature aggregation and misfolding of newly synthesized chains. They also prevent non-productive interactions with other cell components, and direct the assembly of larger proteins and multiprotein complexes. Such proteins also mediate the refolding of previously folded proteins during exposure to cellular stress, and assist newly synthesized proteins in the process of translocation from the cytosol into the mitochondria and the endoplasmic reticulum. Chaperones generally recognize the non-native states of many different polypeptides, primarily by binding to solvent-exposed hydrophobic amino acid stretches, or surfaces that are normally buried inside the protein structures. The protein folding activity of the 70 kDa heat shock protein family is driven by their ATPase activity that regulates cycles of polypeptide binding and release [Liberek, K. et al, (1991) J. Biol. Chem, 266: 14491-14496].

Members of the Hsp70 family are characterized by a multihelical lid assembly over a peptide-binding cavity. Primary sequence alignments of different Hsp70 family members have suggested structural similarities in the C-terminal multihelical lid domain [Bertelsen, E. B. et al, (1999) Protein Sci., 8: 343-354]. Although these regions have low sequence homology, homology modeling indicates that in spite of the amino acid alterations, the general fold of all Hsp70 proteins is very similar. Many residues considered important in structural design are fairly well conserved. Indeed, the conformation of synthetic E. coli and S. aureus D-E helix fragments are almost identical. An exemplary 70 kDa heat shock protein is the E. coli DnaK protein [SEQ ID NO: 10].

Based on small angle X ray scattering, DnaK has a dumbbell shaped structure with a maximum dimension of 112 [Shi, L. et al (1996) Biochemistry, 35: 3297-3308]. The crystallographic structures of the human Hsp70 ATPase domain [Sriram, M. et al, (1997) Structure, 5: 403-414] and the E. coli DnaK peptide-binding domain complexed with a peptide substrate have been solved [Zhu, X. et al, (1996) Science, 272: 1606-1014, incorporated herein by reference]. The secondary structure and dynamics of the 10 kDa C terminal variable domain was also characterized by NMR and comprises of a rigid structure of five helices (named A-E) and a flexible C terminal subdomain of 33 amino acids [Bertelsen et al, cited above].

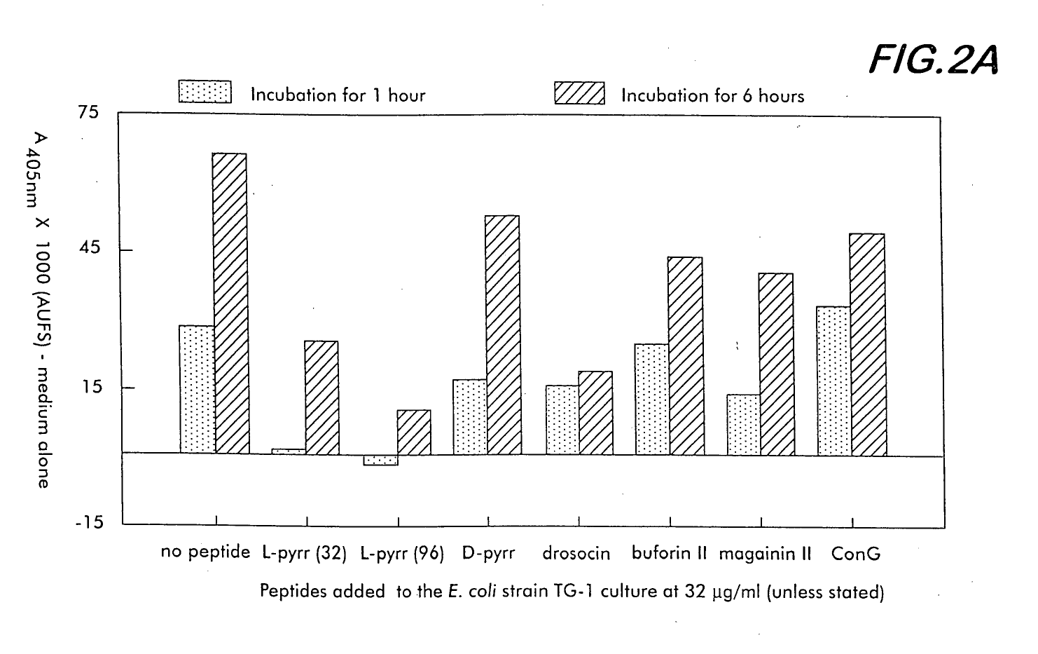

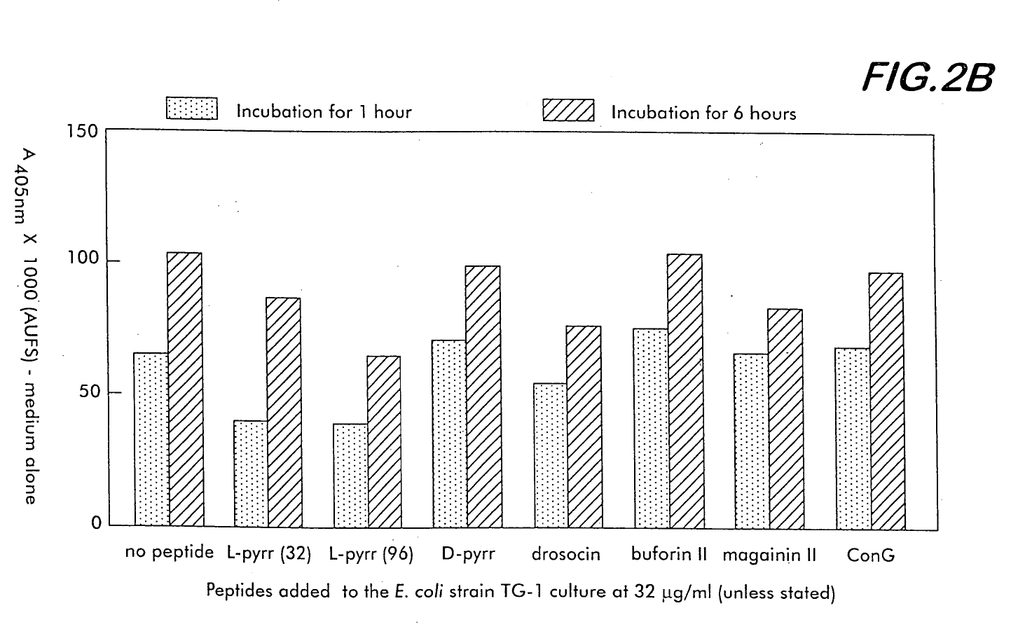

The three-dimensional structure of the C-terminal domain of DnaK, as derived from these X-ray structures is shown in FIG. 2B on page 1608 of Zhu, X. et al, cited above. FIG. 2B shows the structure of the E. coli DnaK from the conventional peptide-binding pocket to the end of helix E. In that figure, the ascending helix on the righthand side is helix A. The transverse helix across the middle of the figure is helix B; the upper transverse helix is helix C. The leftward slanting helix is helix D and the small vertical helix leading to the C terminus is helix E. All references to helices by letter in this specification refer to that published figure.

Frequent opening and closing of the multihelical lid assembly over the peptide-binding cavity is a major means by which the Hsp70 protein family refolds misfolded nascent proteins.

As discussed in the examples below, the inventors determined that the proline-rich antibacterial peptide family drosocin-pyrrhocoricin-apidaecin interact with or bind to the bacterial lipopolysaccharide (LPS) of Gram-negative bacteria and the Hsp70 protein, DnaK, in a specific manner. These same peptides interact with the 60 kDa bacterial chaperonin GroEL in a non-specific manner. Peptide binding to DnaK can be correlated with antimicrobial activity. The antibacterial actions and DnaK-binding can be positively correlated because an inactive pyrrhocoricin analog, made of all D-amino acids, does not interact with DnaK. The inventors thus determined that DnaK is the ultimate target of the pyrrhocoricin-drosocin-apidaecin antibiotic peptides and is not only a temporary player in cell entry and transport processes. Based on comparison with the amino acid sequences of pyrrhocoricin-responsive and pyrrhocoricin-non-responsive bacterial strains, the binding to DnaK takes place between the conventional peptide-binding pocket and the extreme C-terminus of the Hsp.

As further shown in the examples below, pyrrhocoricin and drosocin affect DnaK's two major functions, the ATPase activity and refolding of misfolded proteins. The modification of the ATPase activity was studied with a commercially available recombinant DnaK preparation and direct measurements of phosphate release from ATP. Biologically active pyrrhocoricin made of L-amino acids diminished the ATPase activity of recombinant DnaK. The inactive D-pyrrhocoricin analog, and the membrane-active antibacterial peptides cecropin A and magainin II, each failed to inhibit the DnaK-mediated phosphate release from adenosine 5′-triphosphate (ATP). Drosocin did not influence the ATPase activity.

The protein folding ability was assessed by measuring the enzymatic activity of live bacteria upon incubation with antibacterial peptides. The effect of pyrrhocoricin on DnaK's refolding of misfolded proteins was studied by assaying the alkaline phosphatase and β-galactosidase activity of live bacteria. Both peptides inhibited the DnaK-mediated protein folding as demonstrated by the significant reduction in β-galactosidase and by the less prominent, but still observable, reduction of the alkaline phosphatase activities. Remarkably, both enzyme activities were reduced upon incubation with L-pyrrhocoricin or drosocin. D-pyrrhocoricin, magainin II or buforin II, an antimicrobial peptide involved in binding to bacterial nucleic acids, had only negligible effect.

Pyrrhocoricin's dual actions compared to drosocin's single effect explains the markedly increased bacterial killing potency of the former peptide [Hoffmann, R. et al, (1999) Biochim. Biophys. Acta, 1426: 459-467]. Since both termini of pyrrhocoricin are required to exhibit the antibacterial activity, the inventors determined that these two ends must be covalently connected, as a mixture of the two halves fail to kill bacteria. Competition fluorescence polarization suggested two independent pyrrhocoricin binding sites on DnaK. Based on a comparison of the DnaK sequences of pyrrhocoricin-responsive and pyrrhocoricin non-responsive bacteria, the inventors determined that at least one binding site on DnaK is located between the conventional peptide-binding pocket and the extreme C-terminus of the protein. The hinge region between helices D and E was identified as at least one site where the N-terminus of pyrrhocoricin binds to DnaK. In addition to binding to the multihelical lid, pyrrhocoricin may also interact with the conventional peptide-binding pocket. According to fluorescence polarization and dot blot analysis of synthetic DnaK fragments and labeled pyrrhocoricin analogs, pyrrhocoricin bound with a K_{d} of 50.8 μM to the hinge region around the C-terminal helices D and E, at the vicinity of amino acids 583 and 615 of SEQ ID NO: 10. More specifically, the inventors theorize that pyrrhocoricin is anchored to both the D helix and E helix of E. coli DnaK by salt bridges at R19 of SEQ ID NO: 3 to E590 of SEQ ID NO: 10 and R9 of SEQ ID NO: 3 to E599 of SEQ ID NO: 10. Pyrrhocoricin binds the hinge region by a snug fit of the PRP aa residues 13-15 of SEQ ID NO: 3 to the hinge VSQ aa 594-596 of E. coli DnaK [SEQ ID NO: 10]. This three point interaction prevents movement of the hinge region. Pyrrhocoricin binding was not observed to the homologous DnaK fragment of Staphylococcus aureus, a pyrrhocoricin non-responsive strain. In line with the lack of ATPase inhibition, drosocin binding appears to be slightly shifted towards the D helix.

These experiments clearly demonstrated that a primary binding site of pyrrhocoricin in E. coli DnaK is located in the neighborhood of the hinge between C-terminal helices D and E. As pyrrhocoricin diminished the ATPase activity of recombinant DnaK, the D-E helix region is likely one of those C-terminal domains that allosterically influence the ATPase actions. A weak binding to drosocin was observed with the binding site slightly shifted towards the D helix.

Unlabeled pyrrhocoricin kills bacteria in the mid-nanomolar concentration range, but the activity decreases considerably when the N-terminus is labeled with fluorescein or biotin. Still, the 5 micromolar IC₅₀ of the fluorescein-labeled peptide is below the mid-micromolar binding constant to the DnaK D-E helix hinge fragment. This difference in binding/killing efficacy can be explained on the basis of three different scenarios. First, the antibacterial peptides reach the intracellular milieu by a complex transport mechanism [Castle, M et al, (1999) J. Biol. Chem., 274:32555-32564], which can allow intracellular accumulation of the peptide for effective killing. Second, the antibacterial peptide may bind to full-sized DnaK protein with a considerably higher efficacy than it does to the isolated peptide fragment. If it is indeed true that pyrrhocoricin sees not only the primary sequence, but also the secondary structure of the D-E helix hinge region, it should interact with the full-sized protein much more efficiently. Third, the D-E helix region is just one of the pyrrhocoricin-binding sites on DnaK. According to the examples below, the D-E helix represents a specific binding site of the peptide, but based on non-specific binding spots on the peptide-blot, there are additional pyrrhocoricin-binding sites which could contribute to the efficacious bacterial killing.

Without wishing to be bound by theory, the inventors elucidate a mechanism by which the proline-rich antibacterial peptides kill bacteria by preventing the frequent movements of the multihelical lid over the peptide binding cavity. By permanently closing the multihelical lid over the peptide-binding pocket, the peptides inhibit chaperone-assisted protein folding. The inventors have demonstrated that binding of DnaK by pyrrhocoricin and drosocin, antibacterial peptides isolated from insects, prevents the frequent opening and closing of the multihelical lid over the peptide binding pocket of DnaK, preferably by binding to the D-E helix region. These peptides thus kill the responsive bacterial strains. The biochemical results were strongly supported by molecular modeling of DnaK—pyrrhocoricin interactions.

The mechanism of action of these peptides, and their binding sites to Escherichia coli DnaK enabled identification of a receptor and target sequence for development of a broad range of species-specific biocidal compositions. The characterization of the pyrrhocoricin and drosocin and perhaps apidaecin-binding site on E. coli DnaK identifies the D-E helix hinge and the region around it as particularly desirable targets for the design of strain-specific biocidal (e.g., antibacterial) peptides or non-peptide molecules. Due to the prominent sequence variations of procaryotic and eucaryotic Hsp70 or DnaK molecules in the multihelical lid region (but no general-fold variations), new peptides and peptidomimetics are designed that selectively inhibit the protein folding process in single or closely related bacterial strains, parasites, fungi, insects and rodents. Because this domain is remarkably dissimilar in various bacterial and mammalian DnaK sequences, one of skill in the art may design peptides or non-peptide molecules that selectively kill one species, e.g., a bacterium, without toxicity to experimental animals or humans. The strain-specific biocidal peptides and peptidomimetics which inhibit chaperone-assisted protein folding permits their use in control of the growth and reproduction not only of bacteria, but also fungi, parasites, insects and rodents.

II. Definitions

By the term “biocidal” or “biocidal compound or molecule” as used in this specification is meant a proteinaceous or non-proteinaceous molecule, naturally-occurring or synthetic, that, upon contact with a selected organism has the ability to interfere with and retard the growth and replication of a non-human organism, including the ability to kill the organism. The biocidal molecules of this invention exert an effect by interacting with or binding that organism's heat shock protein, and inhibit the ability of the heat shock protein to mediate proper folding of other molecules essential to the organism. For example, an antibacterial or antibiotic is a biocidal compound effective against bacteria. An anti-fungal is a biocidal compound effective against fungi. An insecticide is a biocidal effective against insects, and so on.

By “organism” as used herein is meant any non-human organism which carries an Hsp70-like heat shock protein, which performs the functions described above. Among such organisms include pathogens such as bacteria, fungi, parasitic microorganisms or multicellular parasites which infect humans and non-human animals. Bacteria of particular pharmaceutical interest include, without limitation, species and strains of Escherichia, Streptococcus, Staphylococcus, Bacillus, Agrobacterium, Salmonella, Enterococcus, Pseudomonas, Haemophilus, Moraxella, Neisseria, Helicobacter, Aerobacter, Borellia, and Gonorrhoeae. Some Gram positive microorganisms of interest are Micrococcus luteus and Bacillus megaterium. Exemplary Gram negative microorganisms include Escherichia coli, Agrobacterium tumefaciens, Bacteriocides gingivalis and Salmonella typhimurium.

Still other organisms are other bacterial pathogens that infect humans include pathogenic gram-positive coccii, such as pneumococci; staphylococci; and streptococci. Pathogenic gram-negative cocci include meningococcus; and gonococcus. Pathogenic enteric gram-negative bacilli include enterobacteriaceae; pseudomonas, acinetobacteria and eikenella; melioidosis; salmonella; shigelIa; haemophilus; moraxella; H. ducreyi (which causes chancroid); brucella; Franisella tularensis (which causes tularemia); yersinia (pasteurella); streptobacillus moniliformis and spirillum. Gram-positive bacilli include listeria monocytogenes; erysipelothrix rhusiopathiae; Corynebacterium diphtheria (diphtheria); cholera; B. anthracis (anthrax); donovanosis (granuloma inguinale); and bartonellosis. Also included are pathogenic anaerobic bacteria that cause diseases including, without limitation, tetanus; botulism; tuberculosis; and leprosy.

Parasites include those organisms that cause pathogenic spirochetal diseases such as syphilis; treponematoses: yaws, pinta and endemic syphilis; and leptospirosis, trichomonas, plasmodial infections such as malaria, and toxoplasmosis.

Other organisms as used herein include those higher pathogen bacteria and pathogenic fungi that cause infections including, without limitation, actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Still other organisms include those microorganisms that cause rickettsial infections, such as Typhus fever, Rocky Mountain spotted fever, Q fever, and Rickettsial pox. Specific fungal targets include a wide variety of Candida and Aspergillis species.

Organisms further include those mycoplasma and chlamydial species that cause such infections as mycoplasma pneumoniae; lymphogranuloma venereum; psittacosis; and perinatal chlamydial infections.

Pathogenic eukaryotic organisms include pathogenic protozoans and helminths and infections produced thereby including amebiasis; malaria; leishmaniasis; trypanosomiasis; toxoplasmosis; babesiosis; giardiasis; trichinosis; filariasis; schistosomiasis. Still other organisms include Pneumocystis carinii; Trichans; and Toxoplasma gondii.

The term organisms include infective agents or vectors of disease which are larger than microorganisms, including nematodes; trematodes, flukes, and cestode (tapeworm), and a wide variety of insects, such as mosquitos, flies, roaches, ants, ticks, bees, wasps, etc.

Similarly, microorganisms or larger organisms that infect or infest plants, particularly plants of agricultural importance are also considered under this definition.

Non-human and preferably, non-primate, animals may also be included in the definition of “organisms” for this purpose, including animals considered to be plant or animal ‘pests’, such as a variety of rodent and mice species, snakes and other such animals.

By “binding” is meant binding is the covalent or non-covalent association between a peptide or non-peptide molecule and the heat shock protein. Examples of binding include ionic, hydrophilic, hydrophobic, stearic, hydrogen bonding, and van der Waals interactions.

III. Methods of Identifying Biocidal Compositions

An aspect of this invention is a method for identifying a compound that has a biocidal effect against a selected non-human organism. The method involves screening from among known or unknown, protein and non-protein, peptide or non-peptide, naturally occurring or synthetic molecules (e.g., chemical compounds, small molecules or proteins/peptides, combinatorial libraries, etc. for test compounds or molecules. The desirable test molecule that binds selectively to a target sequence of a multi-helical lid of a heat shock protein (HSP) of the selected organism (preferably not mammalian and not human). Preferably the HSP is a member of the Hsp 70 family. However, the HSP may be related to GroEL. The binding of the test molecule to the organism's HSP inhibits the protein folding activity of the protein. In some embodiments, the binding physically restrains or restricts essential movement of at least one of the multiple hinge regions flanked by adjacent helices in the HSP. Where it is desirable to identify a composition that does not harm humans or other mammals, the method further involves determining that the test molecule does not bind or restrain the movement of a heat shock protein of a specific mammal exposed to the molecule. These methods involve both screening steps to identify target molecules and assay steps to identify the ability of the molecules to produce the required biocidal effects.

A. Screening Methods

One such screening method involves generating a high resolution, three-dimensional structure of the heat shock protein or a target sequence of an organism that is the desired target of the resulting biocidal composition in a computer-modeling program. A test molecule is selected that binds to the HSP or to a target sequence thereof, and restrains the normal movement of the HSP. As described in more detail below, one such target sequence is an amino acid sequence of a selected organism's HSP that is homologous to the three dimensional structure of the E. coli DnaK protein D-E helix domain sequence: IEAKMQELAQVSQ KLMEIAQQQHAQQQTAGADA [SEQ ID NO:6]. Other examples of such homologous target sequences are discussed in detail below. Still other target sequences meeting this description may be generated for use in screening for biocidal compositions effective against other organisms.

Yet another version of this method involves providing a three-dimensional structure of a multi-helical lid of the HSP of the target non-human organism and generating a computer-identified molecule that specifically binds at least one of the hinge regions of the multi-helical lid of the HSP and/or binds at least two of three helices defining the hinge region. The molecule is then assayed for its ability to restrict the movement at least one of the HSP's hinge regions. Preferably the hinge region immobilized or restricted in movement is the hinge region defined by the D-E helix.

A specific embodiment of the present invention is a method for screening or identifying an antibacterial pharmaceutical or veterinary compound useful in mammals by utilizing the bacterial HSP as a receptor in an appropriate screening assay. This method is accomplished by screening from among known or unknown compounds, a test compound that binds selectively to a bacterial heat shock protein, but does not bind to a homologous heat shock protein of mammalian origin. More specifically, the candidate test compound binds to a target sequence on the bacterial heat shock protein, but not to any sufficiently similar sequence on a mammalian heat shock protein. For example, one bacterial heat shock protein used as the “receptor” for the candidate compound is E. coli DnaK. A candidate compound that binds DnaK is tested for binding to a homologous mammalian protein, such as a human or non-human (animal) heat shock protein. For example, Hsp70 is the human heat shock protein that is homologous to DnaK. If the candidate compound binds DnaK, but does not bind Hsp70, it is a likely antibacterial candidate useful in humans for treatment of Escherichia or other bacterial infection, where the bacteria contain related HSPs. The candidate compound is subsequently screened for antibacterial activity against selected bacteria, e.g., E. coli strains.

Alternatively, the bacterial HSP is the E. coli protein GroEL, and the homologous HSP is Hsp60. The determination that a candidate or test compound selectively binds to the bacterial protein but not the human protein provides a first screen for a desirable antibiotic for humans. The test compound is subsequently screened for its antibacterial activity against bacterial strains, e.g., E. coli strains.

These methods rely on the identification of a heat shock protein of a specific organism, e.g., a specific strain of bacteria, or preferably a specific target sequence thereof. The HSP protein or target sequence of the protein is a stereospecific three-dimensional receptor. As described above, certain biocidal molecules can interact with the HSP receptors, in a strain-specific manner, to achieve their biocidal effect. Thus, strain-specific biocidal compounds are identifiable in assay screens employing as receptors the a selected HSP or target sequences of this invention. Such screening assays may also utilize as a source of test compounds a member of the pyrrhocoricin-apidaecin-drosocin family of peptides or an analog or derivative thereof, and other test compounds.

As one example, an exemplary screening method of this invention involves the following steps. A selected heat shock protein or a target sequence thereof is used in a computer-modeling program that generates a high resolution, three-dimensional structure. A candidate peptide or non-peptide compound is computationally designed or selected to bind to or dock with the heat shock protein in a manner similar to that of a member of the pyrrhocoricin-apidaecin-drosocin family of peptides or an analog or derivative thereof to the E. coli DnaK D-E helix. A candidate compound that has the necessary structural characteristics to permit its binding to the heat shock protein/target sequence three-dimensional structure is computationally evaluated and designed by means of a series of steps. These steps include screening the test compounds, test chemical entities, or test peptide fragments and selecting them for the ability to associate with the heat shock protein or target sequence. One skilled in the art may use one of several methods to screen chemical entities or fragments for their ability to interact with or bind the heat shock protein/target peptide.

This process begins by visual inspection of, for example, a three dimensional structure of the selected heat shock protein, e.g., DnaK, on the computer screen. Selected fragments or chemical entities may then be positioned in a variety of orientations for determining structural similarities, or docked, within the binding site of the heat shock protein.

Specialized computer programs that may also assist in the process of selecting fragments or chemical entities that can interact with the bacterial heat shock proteins/target peptides, include the GRID program available from Oxford University, Oxford, UK. [P. J. Goodford, “A Computational Procedure for Determining Energetically Favorable Binding Sites on Biologically Important Macromolecules”, J. Med. Chem., 28:849-857 (1985)]; the MCSS program available from Molecular Simulations, Burlington, Mass. [A. Miranker and M. Karplus, (1991) “Functionality Maps of Binding Sites: A Multiple Copy Simultaneous Search Method”, Proteins: Structure Function and Genetics 11: 29-34]; the AUTODOCK program available from Scripps Research Institute, La Jolla, Calif. [D. S. Goodsell and A. J. Olsen, (1990) “Automated Docking of Substrates to Proteins by Simulated Annealing”, Proteins: Structure, Function, and Genetics, 8:195-202]; and the DOCK program available from University of California, San Francisco, Calif. [I. D. Kuntz et al, “A Geometric Approach to Macromolecule-Ligand Interactions”, (1982) J. Mol. Biol., 161:269-288], software such as Quanta and Sybyl, followed by energy minimization and molecular dynamics with standard molecular mechanics force fields, such as CHARMM and AMBER. Additional commercially available computer databases for small molecular compounds include Cambridge Structural Database, Fine Chemical Database, and CONCORD database [for a review see Rusinko, A., (1993) Chem. Des. Auto. News, 8:44-47].

Once suitable chemical entities or fragments have been selected, they can be assembled into a single compound. Assembly may proceed by visual inspection of the relationship of the fragments to each other on the three-dimensional image displayed on a computer screen in relation to the structure of the heat shock protein. This would be followed by manual model building using software such as Quanta or Sybyl software. Useful programs to aid one of skill in the art in connecting the individual chemical entities or fragments include the CAVEAT program [P. A. Bartlett et al, (1989) “CAVEAT. A Program to Facilitate the Structure-Derived Design of Biologically Active Molecules”, in Molecular Recognition in Chemical and Biological Problems”, Special Pub., Royal Chem. Soc. 78, pp. 182-196], that is available from the University of California, Berkeley, Calif.; 3D Database systems such as MACCS-3D database (MDL Information Systems, San Leandro, Calif.) [see, e.g., Y. C. Martin, (1992) “3D Database Searching in Drug Design”, J. Med. Chem., 35:2145-2154]; and the HOOK program, available from Molecular Simulations, Burlington, Mass.

An alternative to synthetically preparing a molecule that binds HSP and inhibits its normal protein-folding functions in a step-wise fashion one fragment or chemical entity at a time as described above, inhibitory or other HSP binding compounds may be designed as a whole or “de novo” using either the empty active site, target sequence or optionally including some portion(s) of a pyrrhocoricin or derivative compound. Compounds that mimic a ligand of the heat shock protein are designed as a whole or “de novo” using methods such as the LUDI program [H.-J. Bohm, (1992) “The Computer Program LUDI. A New Method for the De Novo Design of Enzyme Inhibitors”, J. Comp. Aid. Molec. Design, 6:61-78], available from Biosym Technologies, San Diego, Calif.; the LEGEND program [Y. Nishibata and A. Itai, (1991) Tetrahedron, 47:8985], available from Molecular Simulations, Burlington, Mass.; and the LeapFrog program, available from Tripos Associates, St. Louis, Mo. Other molecular modeling techniques may also be employed in accordance with this invention. See, e.g., N. C. Cohen et al, (1990) “Molecular Modeling Software and Methods for Medicinal Chemistry”, J. Med. Chem., 33:883-894. See also, M. A. Navia and M. A. Murcko, (1992) “The Use of Structural Information in Drug Design”, Current Opinions in Structural Biology, 2:202-210. For example, where the structures of test compounds are known, e.g., an analog or derivative of a member of the pyrrhocoricin-apidaecin-drosocin family of peptides, a model of the test compound is superimposed over the model of a known binding peptide of the heat shock protein, e.g., pyrrhocoricin. Numerous methods and techniques are known in the art for performing this step, and any of those methods and techniques may be used. See, e.g., P. S. Farmer, Drug Design, Ariens, E. J., ed., Vol. 10, pp 119-143 (Academic Press, New York, 1980); U.S. Pat. No. 5,331,573; U.S. Pat. No. 5,500,807; C. Verlinde, (1994) Structure, 2:577-587; and I. D. Kuntz, (1992) Science, 257:1078-1082.

The model building techniques and computer evaluation systems described herein are not a limitation on the present invention. Using these computer evaluation systems, a large number of compounds are quickly and easily examined. Consequently, expensive and lengthy biochemical testing is avoided. Moreover, the need for actual synthesis of many compounds is effectively eliminated. The method of this invention permits the identification, design and use of a compound useful as a novel biocidal reagent for a variety of uses, depending upon the identity of the organism that supplied the HSP.

In still anther embodiment of a method of this invention, the heat shock protein receptor and test compound or candidate peptides are employed in a suitable competitive assay method to assess the ability of the test compound to competitively displace a known peptide from binding to a selected heat shock receptor. Depending on the assay selected, the heat shock protein (e.g., E. coli DnaK) to which a selected peptide (e.g., pyrrhocoricin) is known to bind is immobilized directly or indirectly on a suitable surface. As one example, this assay can be conducted using an ELISA format. Suitable immobilization surfaces are well known. As one example, a wettable inert bead is used. As another example, the ligand is bound to a 96 well plate. Thereafter selected amounts of the test compounds are exposed to the immobilized heat shock protein. Those test compounds are selected that can compete with at least one compound that does bind the target sequence of the HSP. Once those test compounds that compete with the known binding compound for binding to the target sequence are identified, they are further screened for anti-pathogenic, antibacterial or anti-fungal activities. It is within the skill of the art to prepare conventional assay formats such as the methods described in the examples below or other assays for identification of test compounds that compete with the peptides of this invention for binding to the receptor.

A suitable specific competitive assay is readily determined by one of skill in the art provided with the teachings herein. For example, a method of this invention for identifying an antibacterial pharmaceutical or veterinary compound includes the steps of performing a competitive assay with (i) a selected HSP of a non-human target organism, (ii) a compound known to bind that HSP, and (iii) a test compound; and (b) identifying the test compound that competitively displaces the binding of the compound (ii) to the HSP. This method specifically employs as a receptor, a heat shock protein, e.g., DnaK or GroEL. The method further comprises the step of testing the candidate compound to ensure that it does not bind a mammalian heat shock protein, and selecting the compound that does not bind to the mammalian heat shock protein. Still another method step includes testing the selected candidate compound in an assay for a suitable antipathogenic, e.g., antibacterial, activity against a selected pathogenic strain. In this manner, strain specific peptides or test compounds can be identified and/or synthesized.

Still other assays and techniques also exist for the identification and development of compounds and drugs that can selectively bind a heat shock protein receptor, and preferably not bind a mammalian heat shock protein receptor. These include the use of phage display system for expressing the heat shock proteins/peptides, and the use of a combinatorial library to produce the peptides for binding studies. See, for example, the techniques described in G. Cesarini, (1992) FEBS Letters, 307(1):66-70; H. Gram et al, (1993) J. Immunol. Meth., 161:169176; C. Summer et al, (1992) Proc. Natl. Acad. Sci., USA, 89:3756-3760, incorporated by reference herein.

Other conventional drug screening techniques use the heat shock proteins and target sequences as receptors for biocidal compounds. As one example, a method for identifying a compound that specifically and selectively binds to a selected heat shock protein includes simply the steps of contacting a selected heat shock protein/peptide sequence with a test compound to permit binding of the test compound to the heat shock peptide; and determining the amount of test compound, if any, that is bound to the heat shock receptor. Such a method may involve the incubation of the test compound and the heat shock protein/peptide immobilized on a solid support. Typically, the surface containing the immobilized heat shock protein/peptide is permitted to come into contact with a solution containing the candidate test compound and binding is measured using an appropriate detection system. Suitable detection systems include the streptavidin horseradish peroxidase conjugate and direct conjugation to a tag, e.g., fluorescein. Other systems are well known to those of skill in the art. This invention is not limited by the detection system used. A similar protocol is employed with the mammalian heat shock protein, e.g., a human or animal protein, to assess the inability of the candidate compound to bind the mammalian protein. Thereafter a conventional assay for the level of bioactivity against the organism permits the final identification of the candidate compound as a suitable biocidal compound for pharmaceutical or other use.

Still another screening or design approach to design novel biocidal compounds or to identify biocidal uses of known compounds involves probing the unbound crystals or binary or preferably ternary crystals of the HSP to establish through structure-based design, small molecule lead compounds composed of a variety of different chemical entities to determine optimal sites for interaction between candidate molecules and the protein. For example, the pyrrhocoricin—E. coli DnaK contact residues are identified by co-crystallizing the peptide and the HSP or its peptide-binding fragment or by using NMR and transferred nuclear Overhauser-effects (or data from other NMR methods) on the same samples. Then small molecules that are predicted from the contact residues and other structural information to bind the desired HSP and function as effective inhibitors of HSP protein folding activity. Such molecules can be synthesized and studied in complex with the protein enzyme by X-ray crystallography. In addition, such molecules can be assayed for their ability to function as effective inhibitors in solution. Molecules that bind tightly can then be further modified and synthesized and tested for their HSP inhibitor activity according to known procedures [J. Travis, Science, 262:1374 (1993)].

Another approach made possible by this invention, is to screen computationally small molecule data bases for chemical entities or compounds that can bind in whole, or in part, to the selected HSP at either a selected binding site or target sequence, e.g., between the D and E helices. In this screening, the quality of fit of such entities or compounds to the binding site may be judged either by shape complementarity or by estimated interaction energy [E. C. Meng et al, J. Comp. Chem., 13:505-524 (1992)].

Thus, the three dimensional structures of the selected HSPs are used to permit the screening of known molecules and/or the designing of new molecules which bind to the HSP structure, particularly at the target sequence homologous to the DnaK sequence, or between two adjacent helices, via the use of computerized evaluation systems. For example, computer modeling systems are available in which the sequence of the HSP, and/or the HSP structure (i.e., atomic coordinates of the HSP, or their complexes and/or the atomic coordinates of the pyrrhocoricin binding active site cavity or other binding sites, bond angles, dihedral angles, distances between atoms in the active site region, etc.), may be input. Alternatively, similar information may be input into computer readable form. Thus, a machine readable medium may be encoded with data representing the coordinates of a selected HSP. The computer then generates structural details of the site into which a test compound should bind, thereby enabling the determination of the complementary structural details of the test compound.

More particularly, the design of compounds that bind to or inhibit the movement of the helices of the multihelical lid of a selected HSP according to this invention generally involves consideration of two factors. First, the compound must be capable of physically and structurally associating with the HSP and, particularly, with the active site between the helices thereof.

Second, the compound must be able to assume a conformation that allows it to associate with the selected HSP. Although certain portions of the compound will not directly participate in this association with the HSP, those portions may still influence the overall conformation of the molecule. This, in turn, may have a significant impact on potency. Such conformational requirements include the overall three-dimensional structure and orientation of the chemical entity or compound in relation to all or a portion of the binding site, or the spacing between functional groups of a compound comprising several chemical entities that directly interact with the HSP.

The potential inhibitory or binding effect of a chemical compound on these sites may be analyzed prior to its actual synthesis and testing by the use of computer modeling techniques. If the theoretical structure of the given compound suggests insufficient interaction and association between it and the HSP, synthesis and testing of the compound is obviated. However, if computer modeling indicates a strong interaction, the molecule may then be synthesized and tested for its ability to bind to the HSP and inhibit its protein folding capabilities, using a suitable assay, such as described in the examples for an anti-bacterial assay. In this manner, synthesis of inoperative compounds may be avoided.

An inhibitory or other binding compound of a selected HSP may be computationally evaluated and designed by means of a series of steps in which chemical entities or fragments are screened and selected for their ability to associate with the individual binding pockets or other areas of the HSP.

One skilled in the art may use one of several methods to screen chemical entities or fragments for their ability to associate with these HSPs and more particularly with the individual binding pockets or clefts of the active site. This process may begin by visual inspection of, for example, the active site on the computer screen based on the crystal coordinates provided herein. Selected fragments or chemical entities may then be positioned in a variety of orientations, or docked, within a binding pocket or cleft of the HSP. Docking may be accomplished using software such as Quanta and Sybyl, followed by energy minimization and molecular dynamics with standard molecular mechanics force fields, such as CHARMM and AMBER.

In another aspect, the known structures of the HSP permit the design and identification of synthetic compounds and/or other molecules which have a shape complementary to the conformation of the HSP target sequence of the invention. Using known computer systems, the coordinates of the HSP structure may be provided in machine readable form, the test compounds designed and/or screened and their conformations superimposed on the structure of the HSP. Subsequently, suitable candidates identified as above may be screened for the desired inhibitory bioactivity, stability, and the like.

B. Assay Steps

According to this invention, the methods for screening the test biocidal compounds heat shock protein, and thus have utility as, e.g., therapeutic biocidal drugs, include both direct assays and indirect assays. The methods of the invention may further involve testing the designed or selected test compound for binding to a mammalian Hsp70 heat shock protein, if the intention is to screen or design a test compound that is noninjurious to the selected mammal, e.g., human. Regardless of whether the three dimensional structure of the HSP or a targeted portion of it is generated by the computer, these methods optionally further involve testing the selected molecule in an assay for in vitro binding to synthetic DnaK fragments. These methods also optionally involve testing the molecule's ability to inhibit protein folding in live cells. Still another optional method step involves testing the ability of the molecule to control the organism population in a suitable in vivo biological assay with the organism, wherein contact by the molecule with the organism retards the growth or reproduction of the organism. Another optional method step for the screen includes further testing the selected molecule for lack of binding to a homologous mammalian, preferably a primate, and more preferably a human, heat shock protein. Suitable assays for conducting these steps are discussed below.

Such assays may use steps now conventional in the art. Direct assays are anti-pathogen assays, e.g., antibacterial assays, that look for the growth inhibitory capacity of the test molecule, and at modifications for optimizing the growth of the new species. An exemplary direct assay is the in vitro assay of Example 2 or the in vivo assay described in Example 5 below. The efficacy of the molecules designed to kill bacteria, parasites and fungi may also be studied in modified versions of the assays of Example 2 or 5.

More preferably, indirect assays are used to test the ability of the test molecule to inhibit protein folding or other activities of the HSP as reflected by live cells. Examples of such indirect assays are the enzymatic assays described in Example 8. Still other enzymatic assays, which demonstrate inhibition of some essential activity of the organism's HSP, may be selected or designed by one of skill in the art without undue experimentation.

Other assays for use in the steps of these methods are challenge assays in which the molecules are tested for their ability to protect experimental animals against bacterial, parasitic or fungal infection, where the molecule is intended for pharmaceutical use. Alternatively, in vivo assays may involve exposing an insect or pest, e.g., flies or mice, to effective amounts of the molecules and scoring the results. In another type of assay, COS cell survival can be studied by counting the infected cells and measuring the rate of proliferation after addition of the test molecules.

For example, in vitro and in vivo assays for antibiotic efficacy and/or metabolic stability that are useful for screening the candidate compounds are selected from among those available and known in the art.

Suitable assays for use herein include, but are not limited to, the assays shown below in the examples to detect the antibacterial effect of peptides, an enzyme-linked immunosorbent assay (ELISA), a fluorescence polarization assay, an ALP or β-galactosidase assay such as those in the examples. However, other assay formats are useful; the assay formats are not a limitation on the present invention.

Once identified and screened for biological activity, these inhibitors may be used therapeutically or prophylactically to block the protein refolding activity of the HSP of the targeted organism. Therefore, the design of small molecule compounds that can be used to inhibit or modulate HSP activity have applications in the treatment of particular infections and in the spread of other diseases by insect or rodent vectors. Additionally, such small molecule inhibitors to specific HSPs are useful experimental reagents for modulating gene expression in clinical and in research settings.

IV. Target Sequences of the HSP

A target sequence or domain of a selected organism's (e.g., a bacterial) heat shock protein that acts as a receptor for ligands that have biocidal activity is identified by the use of homology modeling. Homology modeling relies on the sequence alignment of the target sequence with a selected template sequence, e.g., the D-E helix domain of the E. coli DnaK protein [SEQ ID NO: 6], for which the three-dimensional structure is known. Such modeling is accomplished using, e.g., the SWISS-MODEL [Peitsch, M. C. (1996) Biochem. Soc. Trans. 24: 274-279; Peitsch, M. C., and Guex, N. (1997) Large-scale comparative protein modeling. In: Proteome research: new frontiers infunctional genomics, (Wilkins, M. R., Williams, K. L., Appel, R. O., and Hochstrasser, D. F., eds.) Springer. pp. 177-186] at the Expert Protein Analysis System proteomics server of the Swiss Institute of Bioinformatics (http://www.expasy.ch).

Essentially, using the E. coli DnaK D-E helix domain as a baseline, one prepares from other HSP sequences an amino acid alignment that calculates priority scores to given amino acids compared to similar sequences. When the strict amino acid homologies between protein pairs are established, the alignment is refined based on three dimensional conformational characteristics. The weight-averaged position of each atom in the target sequence is calculated based on the location of the corresponding atoms in the template. This step generates the initial framework for the 3D structure.

Then, loops for which no structural information is available in the template structure are constructed. This step is performed by searching a database, such as the Brookhaven Protein Data Bank (PDB), for fragments which could accommodate onto the framework. Since loop building only adds the C atoms of the target protein, the rest of the backbone must be completed by using a pentapeptide library (PDB). Finally the side chain atoms are constructed based on most probable rotamers. The resulting construct is then energy minimized. Preferably the molecules that bind this domain, do not bind a mammalian heat shock protein. The three dimensional configuration of this target sequence is preferably not found in certain mammalian heat shock proteins, or is not found in a position that is capable of being bound by the biocidal molecule.

For example, such a sequence exists in DnaK, probably located at the carboxy terminus. A similar or homologous sequence having a homologous three dimensional structure is not found in human Hsp70, so that molecules that bind to the D-E helix of E. coli DnaK do not bind to the human Hsp70 D-E helix domain. As another example, such a sequence exists in GroEL, but is not similarly found in human Hsp60. Such target sequences may be used in the above-described screening assays or competitive assays or computerized analyses to identify or design a biocidal compound or molecule.

As discussed above, one such target sequence having a precise three dimensional structure is the D-E helix of DnaK of E. coli. Thus, this target sequence has the 33 amino acid sequence IEAKMQELAQVSQKLMEIAQQQH AQQQTAGADA [SEQ ID NO:6]. Given the overall similarity of the Hsp70 family of peptides, other suitable D-E helix three dimensional target sequences from organisms other than E. coli may be obtained by homology modeling. Thus such target sequences for the HSPs of other organisms may be isolated and used to develop species-specific biocides. Preferably, such other target sequences are homologous to the D-E helix of SEQ ID NO:6, or to a fragment thereof. A desirable fragment includes the first 24 amino acid residues of the above sequence. Other fragments include larger sequences up to the entire 33 amino acid sequence. Still other fragments may have additional amino acids on the N- and C-termini of the above peptide.

More preferably, within species target peptides are identified by also having at least 65% sequence homology to the specific target amino acid sequences identified herein. Such sequence homology for polypeptides, which is also referred to as sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. Unless otherwise specified, the parameters of each algorithm discussed above are the default parameters identified by the authors of such algorithms.

For example, a preferred algorithm when comparing the specific SEQ ID NO:6 to a database containing a large number of sequences from different organisms is the computer program BLAST, especially blastp or tblastn. As an example, preferred parameters for blasp are:

| | |
|---|---|
| Expectation value: | 10 (default) |
| Filter: | seg (default) |
| Cost to open a gap: | 11 (default) |
| Cost to extend a gap: | 1 (default) |
| Max alignments: | 100 (default) |
| Word size: | 11 (default) |
| No. of descriptions: | 100 (default) |
| Penalty Matrix: | BLOWSUM62. |

The length of peptide sequences compared for homology is generally at least about 16 amino acid residues, but can be larger.

Because other Hsp70 family heat shock proteins are found in other species of organisms, homologous target sequences may be obtained and/or located by conventional hybridization or other probing methods using the SEQ ID NO: 6. Alternatively, other homologous sequences may be generated by the above-noted computer programs. Based on this invention, sequences from other bacterial heat shock proteins (that may bind pyrrhocoricin) are useful as targets for screening and identifying other antibacterial compounds. Similarly other heat shock proteins that do not bind pyrrhocoricin may nevertheless be employed as targets in screening assays to identify novel biocidal compounds directed against other organisms.

Thus, one example of an HSP target is SEQ ID NO: 6, a sequence having at least 65% homology thereto, or fragments thereof. A particularly desirable fragment includes the first 24 amino acids of SEQ ID NO: 6, or a sequence having at least 65% homology thereto.

Another example of such an HSP target includes the S. typhiimurium DnaK sequence IEAKMQELAQVSQKLMEIAQQQHAQQQAGSAD A [SEQ ID NO: 26] or smaller fragments thereof, or sequences having at least 65% homology thereto. A desirable fragment comprises the first 24 amino acids of that fragment. Another example of such target HSP sequence includes the A. tumefaciens DnaK sequence IQAKTQTLMEVSMKLGQAIYEAQQAEAGDA SAE [SEQ ID NO: 15] or small fragments thereof, or sequences having at least 65% homology thereto. A desirable fragment comprises the first 24 amino acids of that fragment. Another target fragment includes the H. influenzae DnaK sequence IEAKIEAVIKASEPLMQAVQAKAQQAGGEQPQQ [SEQ ID NO: 16] or small fragments thereof, or sequences having at least 65% homology thereto. A desirable fragment comprises the first 24 amino acids of that fragment.

Yet other target fragments include the S. aureus DnaK sequence IKSKKEELEKVIQELSAKVYEQAAQQQQQAQGA [SEQ ID NO: 22]; the S. pyogenes DnaK sequence MKAKLEALNEKAQA LAVKMYEQAAAAQQAAQGA [SEQ ID NO:23]; or the C. albicans DnaK sequence YEDKRKELESVANPIISGAYGAAGGAPGGA GGF [SEQ ID NO:24]. Smaller fragments of these specific sequences are also encompassed herein as are sequences having at least 65% homology thereto. Desirable fragments comprise the first 24 amino acids of the above-identified fragments.

One of skill in the art would also understand that modifications of these target sequences, e.g., conservative amino acid replacements, and the like may also be made by using conventional techniques.

V. Compositions of the Invention

Another aspect of this invention includes molecules that bind the selected heat shock protein and restrict the essential mobility of the protein, thereby preventing it from accomplishing its protein folding activity. Such molecules may bind to all or a portion of the HSP or active site of the selected HSP or even be competitive, non-competitive, or uncompetitive inhibitors. Once identified and screened for biological activity, these molecules may be used therapeutically or prophylactically to immobilize the HSP and kill or retard the growth of the target organisms.

Compositions of this invention include a peptide or non-peptide molecule that binds to a selected multi-helical lid of the heat shock protein of a selected organism, wherein the protein inhibits the protein folding activity of that protein, and a carrier suitable for the use of the composition. Exposure of the targeted organism to the composition retards the growth and reproduction thereof. Preferably, the molecule used in the composition bind to and physically restrains essential movement of at least one hinge region of the multi-helical lid of the heat shock protein, or restricts movement of multiple hinge regions of the protein flanked by adjacent helices.

Certain candidate or test compounds may be identified, designed or screened by assays or methods of this invention. Such compounds include any peptide or non-peptide that can selectively bind a heat shock protein, but preferably not a homologous human (or other mammalian) heat shock protein. For example, one subset of likely test peptides or antibacterial molecules are members of the pyrrhocoricin-apidaecin-drosocin family of peptides. The methods of this invention provide a ready means for evaluating the antibacterial capability of analogs or derivatives of the peptides of that family. For example, certain co-inventors have recently identified modified pyrrhocoricin peptides, that are described in detail in International Patent Publication No. WO 00/78956, published Dec. 28, 2000, based on U.S. Provisional Patent Application No. 60/154,135, filed Sep. 15, 1999, and incorporated herein by reference. See, also, Example 4 below. Similarly, other modified peptides of this family are designed and screened according to the methods of this invention. Additionally, the methods of this invention provide a ready means for rapidly screening other peptides or molecules not included in this family for antibacterial activity against different bacteria or for other biocidal activity. Desirable candidate peptides for such screening are prepared conventionally by known chemical synthesis techniques. Among such preferred techniques known to one of skill in the art are the synthetic methods described by Merrifield, (1963) J. Amer. Chem. Soc., 85:2149-2154 ; G. B. Fields et al, (1990) Int. J. Pept, Protein Res., 35:161-164; Y. Angell et al, (1994) Tetrahedron Lett., 35:5891-5894, and similar texts. Alternatively, if desired, conventional molecular biology techniques and site-directed mutagenesis are employed to provide desired peptide sequences.

As one embodiment, a peptide compound according to this invention comprises a modified version of the pyrrhocoricin amino acid sequence VDKGSYLPRPTPPRPI YNRN [SEQ ID NO: 3]. This peptide has biocidal activity against E. coli. Other variants having biocidal activity are identified in International Patent Publication No. WO 00/78956, published Dec. 28, 2000. As another embodiment, a biocidal peptide comprises the amino acid sequence VDKGRYLEAPTRPRPERNRK [SEQ ID NO: 7]. This composition has a biocidal effect on Staphylococcus aureus or Microbacillus luteus. These peptide sequences may be modified by means conventional in the art as mentioned above to obtain other biocidal peptides having similar activity or activities directed against other species of organisms.

Other compositions according to this invention may be defined by the ability to bind to a sequence of the protein that is homologous to a target sequence as described in detail above, e.g., the E. coli DnaK protein sequence of SEQ ID NO:6, the other target sequences specifically identified, a sequence at least 65% homologous thereto, as well as smaller fragments thereof.

Still other candidate or biocidal compounds or molecules are antibodies that are capable of selectively binding the heat shock protein, or the target sequences in favor of mammalian heat shock proteins. A suitable antibody is a polyclonal antibody, a recombinant antibody, a monoclonal antibody, a chimeric antibody, a human antibody, a humanized antibody, an antibody or fragment thereof produced by screening phage displays, or mixtures of any of the above antibody types. The state of the art in the antibody field permits the design of all such types of antibodies. This method provides a way to readily screen the antibodies for a binding function indicative of antibacterial action. Antibodies selected by these methods are further screened in conventional assays for antibacterial activity against a battery of bacteria. For example, polyclonal antibody compositions are produced by immunizing a mammal with a selected heat shock protein or target fragment thereof. Suitable mammals include smaller laboratory animals, such as rabbits and mice, as well as larger animals, such as horse, sheep, and cows. Such antibodies may also be produced in transgenic animals. However, a desirable host for raising polyclonal antibodies to a composition of this invention includes humans.

The polyclonal antibodies raised in the mammal exposed to the heat shock protein or fragment are isolated and purified from the plasma or serum of the immunized mammal by conventional techniques. Conventional harvesting techniques can include plasmapheresis, among others. Such polyclonal antibody compositions may themselves be employed as pharmaceutical or veterinary compositions of this invention. Alternatively, other forms of antibodies are developed using conventional techniques, including monoclonal antibodies, chimeric antibodies, humanized antibodies and fully human antibodies. See, e.g., Harlow et al., Antibodies A Laboratory Manual, Cold Spring Harbor Laboratory, (1988); Queen et al., (1989) Proc. Nat'l. Acad. Sci. USA, 86:10029-10032; Hodgson et al., (1991) Bio/Technology, 9:421; International Patent Publication No. PCT/GB91/01554, International Patent Publication No. WO92/04381 and International Patent Publication No. PCT/GB93/00725, International Patent Publication No. WO93/20210. Other antibacterial antibodies that bind to selected heat shock proteins are developed by screening hybridomas or combinatorial libraries, or by the use of antibody phage displays [W. D. Huse et al., (1988) Science, 246:1275-1281] using the polyclonal or monoclonal antibodies produced according to this invention and the amino acid sequences of the heat shock protein or target sequence thereof. Such antibodies, as with the peptides mentioned above, are screened to determine lack of binding to a homologous mammalian heat shock protein and also antibacterial activity in a conventional assay.

Still other compounds or molecules of this invention include those prepared computationally and synthetically. Molecules that bind selectively to a target sequence of a heat shock protein, but preferably do not bind to a homologous heat shock protein of mammalian origin may be employed in a variety of contexts.

A. Pharmaceutical Compositions and Uses

Certain peptide and non-peptide compounds of this invention are identified by the methods described above as biocidal compounds useful against selected disease causing microorganisms, e.g., bacteria, fungi, etc. Still other peptide and non-peptide compounds that are capable of selectively binding to a heat shock protein but not to a homologous mammalian heat shock protein are useful as active ingredients in pharmaceutical and veterinary compositions for the treatment of bacterial infections in humans and other mammals.

Where the selected organism is a mammalian pathogen, and the molecule does not bind to or restrain the mobility of a heat shock protein of the mammal, the molecule may be admixed with a pharmaceutically acceptable carrier suitable for administration to the mammal. Such a pharmaceutical composition may be administered to a mammal to treat the infection. The composition ultimately kills the pathogen or retards its replication in the treatment of infection.

Pharmaceutical or veterinary compositions of this invention can contain effective amounts of these compounds in conventional pharmaceutically acceptable or physiologically acceptable carriers. Suitable pharmaceutically acceptable carriers for use in a composition of the invention are well known to those of skill in the art. Such carriers include, for example, saline, phosphate buffered saline, oil-in-water emulsions and others. The present invention is not limited by the selection of the carrier. Similarly other active agents, such as other anti-pathogenic molecules or conventional antibiotics, such as vancomycin [see, e.g., International Patent Publication No. WO98/40401, published Mar. 10, 1998, incorporated by reference herein] are components of the pharmaceutical or veterinary compositions of this invention.

The pharmaceutical or veterinary compositions are formulated to suit a selected route of administration, and may contain ingredients specific to the route of administration [see, e.g., Remington: The Science and Practice of Pharmacy, Vol. 2, 19^{th} edition (1995)]. The preparation of these pharmaceutically acceptable compositions, from the above-described components, having appropriate pH isotonicity, stability and other conventional characteristics is within the skill of the art.

A method of treating a mammalian pathogenic infection involves administering to an infected mammal an effective biocidal amount of a compound identified by the methods above. The method is useful in the treatment of infection, e.g., such as infection caused by a Gram negative bacterium or a Gram positive bacterium, among the pathogenic organisms recited above.

According to this invention, a pharmaceutical or veterinary composition as described above is administered by any appropriate route. Preferably the route transmits the identified or designed compound directly into the blood, e.g., intravenous injection. Other routes of administration include, without limitation, oral, topical, intradermal, transdermal, intraperitoneal, intramuscular, intrathecal, subcutaneous, mucosal (e.g., intranasal), and by inhalation. One of skill in the art may also readily select a route of administration that is suitable to the infection site. Some specific examples include, without limitation, a topical solution, creme or ointment for application to a local bacterial infection on the skin, a solution or ointment suitable for application to a local bacterial infection of the eye, a solution or spray suitable for application to a bacterial infection of the throat, and a solution suitable for application to a bacterial infection of the gums.

The amount of the antipathogenic compound, selected or designed using the methods above, present in each effective dose is selected with regard to a variety of considerations. Among such considerations are the type of compound (e.g., peptide, non-peptide, chemical, synthetic, etc.), the type and identity of pathogen causing the infection, the severity of infection, the location of the infection (e.g., systemic or localized), the type of mammal, the mammalian patient's age, weight, sex, general physical condition and the like. The amount of active component required to induce an effective antibacterial effect without significant adverse side effects varies depending upon the compound and pharmaceutical or veterinary composition employed and the optional presence of other components, e.g., antibiotics and the like. Generally, for the compositions containing protein/peptide, or fusion protein, each dose contains between about 50 μg peptide/kg patient body weight to about 10 mg/kg. A more preferred dosage is about 500 μg/kg of peptide. A more preferred dosage is greater than 1 mg/kg or greater than 5 mg/kg. Other dosage ranges are contemplated by one of skill in the art. For example, dosages of the candidate antibacterial compounds of this invention are similar to the dosages discussed for other peptide and non-peptide antibiotics. See e.g., International Patent Publication Nos. WO94/05787, WO99/05270, WO97/30082; and French patent Nos. 2733237, 2695392 and 2732345, among others. For example, it has been noted that an antibacterial effect results from administration of a dosage of deglycosylated pyrrhocoricin of less than 25 mgs/kg body weight, or preferably less than 10 mg/kg body weight. Dosages of the non-peptide compounds is readily determined by one skilled in the pharmaceutical arts based upon the bioactivity in an antibacterial assay, such as those of Examples 5 and 6 below.

Initial doses of the compounds of this invention are optionally followed by repeated administration for a duration selected by the attending physician. Dosage frequency depends upon the factors identified above. As one example, dosage ranges from 1 to 6 doses per day for a duration of about 3 days to a maximum of no more than about 1 week. Still other dosage protocols are selected by the attending physician.

B. Other Uses

Other uses of the molecules of this invention depend upon the nature of the organism against which HSP the biocidal molecule is effective. For example, where the origin of the HSP is a selected agricultural plant pathogen or pest and where the molecule does not bind to or immobilize a heat shock protein of a plant or unintended mammal, it may be used in a pesticide. A pesticide composition may be prepared in a carrier suitable for application to or nearby plants, particularly agricultural plants. This composition, when applied to an agricultural plant, is used to kill the pathogen or pest or retard the replication thereof. Preferably, such a composition is intended to bind and immobilize the HSP of a pathogen or pest, such as a plant bacterium, a plant mycobacterium, or a plant parasite.

Where the organism is an insect and the molecule upon contact with the insect has a similar effect on the insect HSP specifically and not on other species HSPs, the molecule may be admixed with a carrier suitable for use in an insecticide. Application of the insecticide by conventional means, e.g., spraying, liquid application, powder, etc, is used to kill the insect or retards the reproduction and growth thereof, without harm to other plant and mammalian species.

Similarly where the organism is a selected mammalian pest species, such as a mouse, a rodent, etc. and the molecule does not bind to or restrict the essential movement of a primate heat shock protein, specifically a human HSP or HSPs of domestic or farm animals, the molecule is admixed with a carrier suitable for use in a pesticide. Such a pesticide may be formulated in a conventional admixture and applied conventionally in baits and/or traps. This composition upon contact with the pest species, kills the pest or retards the reproduction and growth thereof, without harm to unintended species.

These compositions may appropriately be employed in the treatment of disease and disease vectors for both animals and plants, and in methods for eliminating pests by administering or applying these compositions as one would other compositions of their type.

One of skill in the art can readily determine other uses based on the selection of the organism and the determination of its binding specificity to the organism's HSP but not the HSP of other species.

VI. Molecules that Penetrate the Bacterial Cell Wall

Molecules or compounds that penetrate the peptidoglycan layer of a bacterial cell wall can be constructed from a peptide selected from the pyrrhocoricin-apidaecin-drosocin family and a derivative or analog thereof that binds to the HSP or DnaK present in the lipopolysaccharide layer of Gram-negative bacteria. That peptide is covalently linked to a second compound that has a biological activity within the cell. Methods for making these compounds and for using them in pharmaceutical or veterinary compositions for the treatment of bacterial infections are also part of this invention. Still another aspect of the invention engendered by the discovery that a heat shock protein is the receptor protein of pyrrhocoricin is a molecule that penetrates the peptidoglycan layer of a bacterial cell wall. Gram-negative strains have a cell peptidoglycan wall that is thinner than that of Gram-positive bacteria. However, the cell wall of Gram negative bacteria also contains an outer membrane, composed of a lipid bilayer, some proteins and lipopolysaccharide (LPS), that lies above a layer formed of peptidoglycan with teichoic acid polymers dispersed throughout the layer. The acidic character of the peptidoglycan cell wall naturally binds the highly positively charged antibacterial peptides. As predicted from their positive charge, many antibacterial peptides also bind the negatively charged LPS [Vaara, M. (1992) Microbiol Rev., 56: 395-341]. This seems very beneficial because antibacterial activity of certain peptides must be initiated at the bacterial cell surface if the peptides are too large to diffuse across the outer membrane. Nevertheless, the general destabilization of the outer membrane and the ensuing internalization of some positively charged peptides do not necessarily result in killing the microorganisms without additional intracellular effects.

According to the present invention, a molecule that is capable of penetrating the peptidoglycan of Gram negative or Gram positive bacteria comprises a “transport” peptide of the pyrrhocoricin-apidaecin-drosocin family, or a derivative or analog thereof. Preferably the peptides of this family also bind to the heat shock protein. Preferably, the heat shock protein is E. coli DnaK. Alternatively, the transport peptides bind the LPS of Gram negative bacteria. This transport peptide is covalently linked to a second compound (peptide or non-peptide) that has a desired biological activity within the cell. This covalently linked conjugate compound is capable of penetrating the peptidoglycan wall due to the peptide (i.e., pyrrhocoricin or other peptide or derivative of that family). Once in the bacterial cell, the pyrrhocoricin can perform its antibacterial function and the second compound can perform its function.

The second compound includes other antibacterial peptides or nonpeptide antibacterial compounds, or other compounds that perform a desired effect within the cell, such as an effect on vital cell activity. One of skill in the art of microbiology and/or bacterial infections can select the second compound from among known compounds having the desired bioactivity in the bacterial cell. For example, examples of such second compounds include labels, such as dyes, sequences encoding fluorescent proteins or enzymes which interact with other substrates to produce a signal. Such labels are conventional and may be readily selected. The second compound may also be a gene encoding a therapeutic amino acid sequence, or a sequence missing from the targeted cell. Still another class of second compounds may be desirably lethal to the cell, such as toxins or metabolic poisons and the like. Preferably the second compound is non-toxic to the human or animal cells. This molecule is useful in methods for studying the effects of many types of second compounds upon the bacterial cell. Thus, selection of the second compound is not a limitation on this aspect of the invention. By its conjugation to pyrrhocoricin or a like peptide of the above defined family, the second compound is targeted within the bacterial cells and thus will have its effect on the bacterial cell only and not on or within other cells of the mammal to which the peptide conjugate is administered.

The peptide conjugate is prepared by conventional methods of chemical peptide synthesis by covalently linking the second compound to the “transport” peptide of the pyrrhocoricin, drosocin and apidaecin family or a peptide fragment thereof, or an analog or derivative thereof. See conventional techniques described in Merrifield, (1963) J. Amer. Chem. Soc., 85:2149-2154, among other texts.

Thus, the invention also provides a pharmaceutical or veterinary composition that contains the conjugate in a physiologically acceptable carrier. This composition is useful for the treatment of a bacterial infection in a human or animal. The pharmaceutical composition may further contain any or all of the components described above for the antibacterial pharmaceutical compositions of this invention, and is administered in similar fashion. Such a composition is used to treat a mammalian subject (i.e., human or animal) with a bacterial infection by administering an effective amount of the conjugate to the mammal. Routes of administration and dosages are selected by one of skill in the art with regard to the considerations identified above in the description of antibacterial pharmaceutical compositions of this invention.

VII. Computer Programs

As another aspect of this invention, a computer program is provided that performs the computational analyses described above to permit the design or selection of a biocidal molecule to fit within the three dimensional structure of the selected HSP. More particularly, the program would perform the calculations necessary to design or select a molecule to fit within the hinge region defined by helices D and E of an HSP homologous to E. coli DnaK. More specifically, the computer program is designed to record, sort and calculate the parameters of the programs provided above and to obtain the necessary analytical results. In a preferred embodiment, this computer program is integrated into an analysis instrument, e.g., an X ray apparatus. In still other embodiments, the program is on a separate computer, which is a “plug-in” device for attachment to the analysis instrument. Still another embodiment of this invention is a computer program that is present on a standalone computer, into which data from the instrument is fed. Alternatively, the method of this invention can be generated by use of conventional spreadsheet programs on standalone personal computers. Thus, the program preferably performs all of the calculations necessary to perform the screening methods of this invention by analyzing the data on the test compounds, target sequences and HSP structures.

The following examples illustrate various aspects of this invention. These examples do not limit the scope of this invention which is defined by the appended claims.

### EXAMPLE 1

### Identification of the Target Protein of Pyrrhocoricin

The identification of the target protein was accomplished using four primary steps.

A. Isolation of the Target Protein by Immunoaffinity Chromatography from an E. coli Lysate

In early assays, it was determined that biotin-K-pyrrhocoricin, a molecule represented by the formula: biotin-Lys-Val-Asp-Lys-Gly-Ser-Tyr-Leu-Pro-Arg-Pro-Thr-Pro-Pro-Arg-Pro-Ile-Tyr-Asn-Arg-Asn [SEQ ID NO: 12], kills E. coli strains (including TG-1, or K-12) in the submicromolar range. Based on this, the target protein was isolated from an E. coli lysate with the help of the labeled peptide, that is useful also to purify the complex through the attached biotin. For this latter purpose, an immobilized anti-biotin antibody was used rather than streptavidin derivatives because of the generally observed lower background with anti-biotin monoclonal antibodies (mAbs). The antigen was detached from the antibody in an acidic buffer, and the resulting peptide-target mixture was submitted to SDS-gel electrophoresis, followed sequencing by mass spectroscopy.

The following immunoaffinity purification protocol was used. French pressed E. coli TG-1 (K-12) cell lysates (50 ml) were centrifuged at 2500 rpm for 20 minutes to remove residual cells and cell wall. Four-and-a-half ml bacterial supernatant was mixed with 150 μg of biotin-K-pyrrhocoricin peptide diluted in 1 ml phosphate buffered saline (PBS) and the mixture was incubated at room temperature for 3 hours followed by centrifugation at 2000 rpm for 20 minutes. Anti-biotin mAb (clone BN34) coupled to agarose was washed with PBS to remove NaN₃, and the peptide-lysate target mixture was loaded onto the column. The column was extensively washed with PBS. The target was eluted with five column volumes of 0.1 M glycine (pH 2.9) and the eluant was immediately neutralized with 1 M Tris-HCl (pH 8.0). One ml fractions were collected and the fractions were analyzed for the presence of pyrrhocoricin-binding proteins by 12% SDS-PAGE and Western blot.

The fractions from the immunoaffinity purification showed proteins binding to biotin-K-pyrrhocoricin in diverse amounts and purities. While the cleanest fractions did not seem to contain enough proteins for sequencing, one fraction that contained a number of proteins in lower quantities, appeared to have two proteins in higher amounts, apparently suitable for mass spectroscopy. These two proteins exhibited molecular weights around 60-70 kDa, when transferred to polyvinylidene difluoride (PVDF) membrane and stained with 0.1% amido black 10B.

B. Identification of Pyrrhocoricin-Binding E. coli Proteins by Mass Spectroscopy

The eluted fractions were submitted to another round of SDS-PAGE analysis, designed to yield protein preparations suitable for ensuing sequencing. To this end, the gel was stained using colloidal Coomassie blue. This staining is less sensitive than amido black. However, only those proteins that are present in the gel in quantities suitable for sequencing show positive staining with colloidal Coomassie. None of the fractions from the immunoaffinity column could be stained except the two 60-70 kDa bands from the above-mentioned fraction. These bands were collectively excised from the gel together with a blank portion of the gel and subjected to in-gel tryptic digestion. The resulting peptides were extracted from the gel and purified using a C₁₈ cartridge. The peptide containing fractions were collected and analyzed by Nanospray-ES-MS (electrospray mass spectroscopy). This analysis resulted in four doubly-charged signals, potentially corresponding to E. coli proteins. These were at 923 [M+2H]²+, 889 [M+2H]²⁺, 799 [M+2H]²⁺, and 1220 [M+2H]²⁺, representing four peptide fragments, respectively:

GroEL aa328-345: Asp-Thr-Thr-Thr-Ile-Ile-Asp-Gly-Val-Gly-Glu-Glu-Ala-Ala-Ile-Gln-Gly-Arg (peptide 1) [SEQ ID NO: 13] and

GroEL aa204-219: Phe-Ile-Asn-Lys-Pro-Glu-Thr-Gly-Ala-Val-Glu-Leu-Glu-Ser-Pro-Phe (peptide 2) [SEQ ID NO: 14] [Venner, T. J. and Gupta, R. S., (1990), Biochim. Biophys. Acta, 1087:336-338];

DnaK aa453-467 of SEQ ID NO: 10 (peptide 3) and

DnaK aa322-345 of SEQ ID NO: 10 (peptide 4) [Seaton, B. L. and Vickery, L. E., (1994), Proc. Natl. Acad. Sci. USA, 91:2066-2070].

These peaks were submitted to MS/MS sequencing. As one example, the MS-MS sequence of the doubly charged signal observed at 1220 [M+2H]²⁺ in the nanospray mass spectrum, identified the partial sequence of Ser-Val-Ser-Asp-Leu/Ile-Asp of tryptic peptide 4 [SEQ ID NO: 17]. The sequencing also identified probable amino acid stretches Thr-Ile/Leu-Ile/Leu-Asp-Gly-Val of peptide 1 [SEQ ID NO: 18], Glu-Leu/Ile-Glu-Ser of peptide 2 [SEQ ID NO: 19], and Phe-Asn-Leu-Leu/Ile-Asp-Gly of peptide 3 [SEQ ID NO: 20]. All four partial sequences match the corresponding proposed protein fragments. These experiments clearly identified GroEL and DnaK as proteins strongly binding to biotin-K-pyrrhocoricin.

C. Characterization of the Binding of the Identified proteins to Labeled Pyrrhocoricin by Western-Blotting on the Solid-Phase

Fifty μl aliquot of each fraction from the immunoaffinity column was mixed with 50 μl Laemmli sample buffer (Bio-Rad). Five percent 2-mercaptoethanol was added, and the mixture was boiled for 3 minutes. Ten μl of the boiled samples were processed in a 12% SDS-PAGE at 100 V for 1.5 hours at room temperature. The proteins from the gel were transferred to a nitrocellulose membrane that was equilibrated with 25 mM Tris, and 192 M glycine buffer containing 20% methanol at 100 V for 2 hours at 4° C. The membrane was blocked with 5% milk in a phosphate buffered saline-0.5% Tween 20 buffer (PBST) overnight at 4° C.

The membrane was incubated with 10 ml of 10 μg/ml biotin-K-pyrrhocoricin peptide dissolved in PBST containing 1% bovine serum albumin at room temperature for one hour. After incubation, the membrane was extensively washed with PBST. Streptavidin conjugated to horseradish peroxidase (HRP) (Gibco-BRL) dissolved in 1% BSA-PBST was added to the membrane and was incubated with it at room temperature for 45 minutes. After extensive washing with PBST, the membrane was treated with chemiluminescence luminol-oxidizer (NEN) for one minute. The created chemiluminescence was exposed to a X-Omat blue XB-1 film (Kodak) for 10 seconds, and the film was developed.

The resulting gels showed that the biotin-K-pyrrhocoricin peptide labeled the 60-70 kDa bands strongly. Two additional bands, one running with the front, and another, running close to the 15 kDa molecular weight marker, were also labeled with the peptide. The former band may represent the labeled peptide itself, that was also eluted from the immunoaffinity column. According to the amido black-stained gel, the 15 kDa band did not represent proteinaceous material.

To determine whether isolated heat shock proteins bind to the biotin-K pyrrhocoricin peptide in identical Western-blotting conditions, a number of commercially available eucaryotic and procaryotic heat shock proteins were used: the bacterial chaperonins GroEL (60 kDa) and GroES (15 kDa), and three heat shock proteins, DnaK (70 kDa), DnaJ (40 kDa) and GrpE (25 kDa). These proteins are involved in protein folding during the travel of nascent proteins from the ribosomes to GroEL. In addition, two mammalian heat shock proteins, Hsp60 (the human equivalent of GroEL) and Hsp70 (the human equivalent of DnaK) were used in this experiment to gain insight on why pyrrhocoricin kills bacteria but is not toxic to healthy mice. All these proteins were expressed or overexpressed in E. coli. As a negative control, the guanyl-nucleotide binding protein Ras (21 kDa), also expressed in E. coli was used. Between 1 to about 2.8 μg of each of these proteins was loaded onto 12% SDS-PAGE and the PVDF membrane was stained with amidoblack. The test proteins showed single bands in the expected MW range with approximately equal intensities: Ras (negative control) at 21 kDa; GroES at 15 kDa; GrpE at 25 kDa; DnaJ at 40 kDa; GroEL at 60 kDa; Hsp60 at 60 kDa; DnaK at 70 kDa; Hsp70 at 70 kDa and fraction spanning about 7 kDa to about 80 kDa.

When tested for peptide binding, of the bands that could be stained with amido black, only the heat shock protein DnaK bound to biotin-K pyrrhocoricin. The rest of the proteins showed very weak peptide binding, and can be considered non-binders. The DnaK preparation, however, had two additional nonproteinaceous bands that bound to the labeled pyrrhocoricin. The Ras preparation also had a non-proteinaceous peptide binding band. All of these contaminating bands exhibited molecular weights similar to the additional non-proteinaceous peptide binding bands of fraction from the immunoaffinity purification.

A control peptide-blot was run in which an unrelated biotin-labeled peptide, biotin-GPKG- -tubulin 434-445 was used as the “primary antibody”. This peptide served as a negative control because it is highly negatively charged and does not share any sequence homology to the insect antibacterial peptides. In this blot, the very low molecular weight bands were stained from the eluted fraction and the DnaK preparation together with a near-DnaK band from the early fraction. A low MW band from the Ras preparation, running with the front, was also stained. All of the bands represent unspecific binding.

All of these studies confirmed that DnaK is the bacterial protein target of pyrrhocoricin, because DnaK binds strongly bound to the peptide. It was clear that the peptide also binds an unidentified component running at 15-20 kDa, and to non-proteinaceous components of bacterial preparations. Significantly, the peptide failed to bind Hsp70, the human equivalent of DnaK. This latter observation fully supported in vitro and in vivo antibacterial studies that had showed that pyrrhocoricin kills bacteria without being toxic to isolated mammalian cells or live mice.

Many cationic antibacterial peptides bind the negatively charged LPS of Gram-negative bacteria [Groisman, E. A. (1996) Trends Microbiol., 4:127-128], and this experiment suggested that the non-proteinaceous pyrrhocoricin-binding bands might be bacterial LPS. This theory was further supported by the electrophoretic mobility pattern of E. coli LPS, that exhibits two stronger bands at low MW regions, and a smear of higher MW bands [Inzana, T. J., and ApicelIa, M. A. (1999) Electrophoresis, 20: 462-465]. The location of these bands appeared to be strikingly similar to the two low MW bands on the Western-blot, as well as to the additional unidentified pyrrhocoricin-active bands nearby DnaK. E. coli LPS as well as LPS from S. typhimurium were tested for binding to biotin-K-pyrrhocoricin on Western-blot. In the experimental condition used, the peptide did not label LPS bands when these were nitrocellulose membrane-bound. Thus, the evidence indicated that pyrrhocoricin has a proteinaceous target in bacteria, DnaK, and also binds to two unidentified low MW nonproteinaceous components, albeit with considerably lower efficacy.

Antimicrobial activity was correlated with DnaK binding by testing 1 μg amounts of DnaK and GroEL proteins for binding to biotin-K-pyrrhocoricin, biotin-K-all-D-pyrrhocoricin and biotin-GPKG- -tubulin 434-445 on the peptide blot. While native pyrrhocoricin made from all L-amino acids kills E. coli D22 in nanomolar concentrations, a pyrrhocoricin analog made of all D-amino acids is completely inactive [Otvos et al, 2000, Protein Science, 9:742-749, incorporated herein by reference]. On the blot, the L-peptide bound strongly to DnaK, but the all-D-peptide bound only very weakly. Tubulin bound not at all. These experiments confirmed that killing of bacteria and DnaK binding are positively related events.

D. Characterization of Binding in Solution by Fluorescence Polarization.

All solid-phase assays that separate the bound form of the ligand from the free form are suspect. Therefore, in the next step, the binding of labeled peptides to the heat shock/chaperone proteins and to LPS was observed by fluorescence polarization.

In one example, three fluorescein-labeled peptides were synthesized with the fluorescein label attached at the N-terminus of the peptide: fluorescein-K-pyrrhocoricin (N-F-pyrrhocoricin), fluorescein-K-drosocin (unglycosylated; N-F-drosocin), and fluorescein-K-apidaecin (N-F-apidaecin). A C-terminally labeled peptide was also made, i.e., pyrrhocoricin-K-fluorescein (C-F-pyrrhocoricin). The C-terminally labeled pyrrhocoricin peptide (C-F-pyrrhocoricin) was made to investigate the possibility of spatial separation of the active sites. From earlier experiments, it was clear that pyrrhocoricin and drosocin bind to the receptor(s) with their two terminal domains [Hoffmann, R. et al, (1999) Biochim. Biophys. Acta, 1426: 459-467; McManus, A. et al, (1999) Biochemistry, 38: 705-714].

During fluorescence polarization, positive signals are detected only when the free rotation of the fluorescein attached to one of the interacting partners is slowed down due to binding to the other partner when this label is not exceedingly far from the site of interaction. If the label is placed too far from the binding site, the flexibility of peptide-like structures will resume free rotation of the fluorescein moiety, resulting in no polarization anisotropy, even if positive binding occurs. As a negative control fluorescein-labeled peptide, a fragment of the P-subunit of human tubulin was used [Otvos, L., Jr. et al, (1998) Protein and Peptide Lett., 5: 207-213]. The tubulin fragment was selected to serve as a negative control because it is highly negatively charged and does not share any sequence homology to the insect antibacterial peptides.

The same heat shock proteins and LPS preparations were used as in the Western-blotting, except DnaJ was not studied. Ras was used as a negative control protein. The fluorescein-K-pyrrhocoricin—DnaK binding study was repeated with an additional DnaK preparation, purchased from another source. The labeled peptides were used in fixed 1 nM concentrations. The initial concentration of the proteins was 4 μM, and serial dilutions by two were done until the protein did not bind in at least two dilutions. The 4 μM protein concentration is just barely below the lethal dose of the peptide, and likely represents the raising stretch of the dose-response curve. The initial concentration of LPS was set to 0.5 mg/ml, and dilutions were made until 0.031 mg/ml. This concentration range roughly equals that used for the heat shock proteins.

The N-terminally fluorescein-labeled pyrrhocoricin peptide, K-pyrrhocoricin (also referred to as “fluorescein-K pyrrhocoricin” or “N-F-pyrrhocoricin” in this specification) has the formula: fluorescein-Lys-Val-Asp-Lys-Gly-Ser-Tyr-Leu-Pro-Arg-Pro-Thr-Pro-Pro-Arg-Pro-Ile-Tyr-Asn-Arg-Asn [SEQ ID NO: 25]. 1 nM K-pyrrhocoricin bound to DnaK with 50% higher millipolarization values over the background. From the limited number of data points available, a K_{d} value of approximately 1.1 μM was calculated, confirming the data of the sequencing and the Western-blot.

In solution the binding of fluorescein-K-pyrrhocoricin (in 1 nM concentration) was measured to heat shock proteins (GroEL, Hsp60, DnaK, Hsp70, GroES and Grp E, with Ras as the positive control) at two concentrations (4 μM and 2 μM). The blank showed a minipolarization value of about 62. At both concentrations of Ras, Hsp70, GroES and GrpE, minipolarization values were under 62. For 4 μM Gro EL, the value was over 100, for 2 μM Gro EL, the value was about 85. For 4 μM Hsp60, the value was about 90, for 2 μM Hsp60, the value was about 62. For 4 μM DnaK, the value was about 100, for 2 μM DnaK, the value was about 95. These results demonstrated no binding for GroEL or DnaK at or below 0.5 μM concentration. The peptide did not bind to Hsp70, GroES, GrpE or the negative control Ras (see Table 2). In solution, the peptide did strongly bind to GroEL (with millipolarization values similar to DnaK) and less strongly to Hsp60 (Table 2). The interaction of pyrrhocoricin with GroEL verified the sequencing data. All these findings paralleled those of the solid-phase assay (nitrocellulose membrane-bound proteins).

The reason why GroEL did not bind the peptide in the solid-phase assay likely lies in the nature of the interaction of GroEL with its ligands. GroEL consists of two heptameric rings of 57 kDa subunits that have a three-domain structure [Braig, K. et al, (1994) Nature, 371: 578-586]. The apical domain forms the opening of the cylinder and exposes a number of hydrophobic amino acid residues towards the center that are thought to interact with complementary surfaces of the polypeptide substrate. The intermediate segments allow a hinge-like opening and considerable twisting of the apical domains about the domain junctions [Roseman, A. M. et al, (1996) Cell, 87: 241-251]. Mini-chaperones made of the polypeptide-binding fragments of GroEL assume the same folding pattern as in the full-size molecule [Buckle, A. M. et al, (1997) Biochemistry, 94: 3571-3575], suggesting that the three dimensional structure and the orientation of the hydrophobic amino acids are necessary for efficient ligand binding. Both the denaturing conditions during SDS-PAGE and the binding to the nitrocellulose membrane via the solvent-exposed hydrophobic amino acids can easily eliminate the GroEL—ligand interaction.

In this regard, it is promising that DnaK did not lose its ability to bind pyrrhocoricin on the Western blotting solid-phase. This suggests that the binding of DnaK to pyrrhocoricin is not dependent upon the global fold of the protein, and at least one peptide-binding site lies outside the conventional peptide-binding domain of DnaK. The peptide binding site is identified by the synthetic fragments of DnaK.

Alternatively, while in the case of the multimeric GroEL denaturation is inevitable, for some proteins, a partial restructuring can occur on the nitrocellulose membrane when exposed to certain buffers. Moreover, massively parallel solid-phase screening techniques, such as peptide arrays, can be used.

TABLE 2
Binding of heat shock proteins and lipopolysaccharides to
fluorescein-labeled peptides.

| Protein or | N—F-³ | C—F- | N—F- | N—F- | |
|---|---|---|---|---|---|
| LPS | pyrrhocoricin | pyrrhocoricin | drosocin | apidaecin | N—F-tubulin |
| Ras | − | − | − | − | − |
| GroES | − | not tested (NT) | NT | NT | NT |
| GrpE | − | NT | NT | NT | NT |
| GroEL | ++² | ++ | ++ | + | + |
| Hsp60 | + | NT | NT | NT | NT |
| DnaK | ++/++¹ | ++ | ++ | ++ | − |
| Hsp70 | − | NT | NT | NT | NT |
| E. coli LPS | ++ | + | ++ | ++ | − |
| S. typhimurium | +++ | +++ | +++ | +++ | − |
| LPS | | | | | |

¹Studied with three different DnaK preparations (Sigma, Accurate, and StressGen).
²Studied with two GroEL preparations (Sigma and StressGen)
³N—F or C—F indicates the position of the fluorescein label on the N- or C-terminus.

The LPS preparations bound to the N-terminally labeled pyrrhocoricin peptide very strongly (Table 2). Little decrease in binding efficacy was detected at as low LPS concentration as 31 μg/ml (calculating with a MW of 20 kDa, this corresponds to 1.5 μM). The strong binding of DnaK or the two LPS preparations to pyrrhocoricin appeared to be specific for the peptide sequence.

A graph was plotted (not shown) showing the binding of the fluorescein-labeled peptides: C-F-pyrrhocoricin (K-pyrrhocoricin labeled with fluorescein on its C terminus), N-F-pyrrhocoricin, N-F-drosocin, N-F-apidaecin and N-F-tubulin (all at 1 μM concentration) to either E. coli lipopolysaccharide (LPS) or S. typhimurium LPS at various concentrations measured in μg/ml. The N-F-tubulin curves for both types of LPS overlay each other at under 40 millipolarization. A similar graph was plotted (not shown) showing the binding of fluorescein labeled peptides: C-F pyrrhocoricin, N-F-pyrrhocoricin, N-F-drosocin, N-F-apidaecin and N-F-tubulin (all at 1 nM concentration) to heat shock proteins DnaK and GroEL, and to negative control Ras in varying concentrations (μM). These graphs demonstrated that neither the heat shock protein nor the lipopolysaccharides bound to the negative control fluorescein-labeled tubulin peptide (Table 2). In contrast, GroEL did bind the tubulin sequence, with 50% over the background at 4 μM protein concentration, a level comparable to pyrrhocoricin binding. This suggests that GroEL recognized a generally unstructured peptide chain (at least in comparison with well-structured native proteins) carrying a bulky hydrophobic appendage.

Accordingly, GroEL does not seem to be the final bacterial protein target of the short, proline-rich, insect antimicrobial peptides. Rather, it may play a role in the intermediate steps of the sequential molecular interaction cascade of the bacterial cell entry and killing by this peptide family [M. Castle et al, J. Biol. Chem., 274:32555-32564]. In addition, the weak pyrrhocoricin binding to the human equivalent Hsp60 is unlikely to occur without fluorescein addition, eliminating concerns of the therapeutic use of pyrrhocoricin analogs in humans. In support, a small redshift (1 μm) of the 200 μm negative circular dichroism band was detected in both water and 2% octyl-glucoside solutions when the fluorescein-K-N-terminal label was attached to pyrrhocoricin, suggesting altered conformation upon fluorescein addition.

The labeled peptide bound to the commercially available E. coli and S. typhimurium LPS according to fluorescence polarization, but did not bind according to the Western blot. Those biopolymers/proteins that showed strong binding to the N-terminally labeled pyrrhocoricin (DnaK, GroEL, E. coli LPS and S. typhimurium LPS) were tested for their binding to the C-terminally labeled pyrrhocoricin peptide as well as to N-terminally labeled drosocin and apidaecin. The binding pattern of these biopolymers to all three labeled peptides were very similar to that observed with the N-terminally labeled pyrrhocoricin.

In another competition fluorescence polarization with heat shock proteins against labeled and unlabeled pyrrhocoricin, 4 μM DnaK, GroEL or Ras were pre-mixed 4 μM unlabeled pyrrhocoricin and after a 20-minute incubation the N-terminally-labeled fluorescein-K-pyrrhocoricin analog was added in 1 nM concentration. The fluorescence anizotropy was recorded (Table 2). The background reading (without any unlabeled peptide or protein) was 46±7 millipolarization units. In the presence of 4 μM Ras, this value was 52±7. As preincubation with 4 μM and 8 μM pyrrhocoricin decreased the Ras readings with 16 and 27 millipolarization units respectively, the readings for DnaK and GroEL were corrected with these values. The negative control peptide was Conantokin G-Ala7 [L.-M. Zhou et al, (1996) J. Neurochem., 66:620-628], which is similar in size to pyrrhocoricin (17 amino acid residues). In contrast to the positively charged pyrrhocoricin which has a middle—pleated sheet domain, Conantokin G-Ala7 is negatively charged and devoid of any extended structure. Accordingly, unlabeled pyrrhocoricin could, but Conantokin G-Ala7 could not compete for labeled pyrrhocoricin binding, as reported in Table 3 below. Apparently, both the C- and N-termini of pyrrhocoricin were involved in binding to DnaK.

TABLE 3

| | Millipolarization after preincubation with: | | | | |
|---|---|---|---|---|---|
| | pyrrhocoricin | | Conantokin G-Ala7 | | No peptide |
| Protein | 4 μM | 8 μM | 4 μM | 8 μM | — |
| GroEL | 62 | 62 | 75 | 67 | 70 ± 10 |
| DnaK | 110 | 84 | 127 | 114 | 126 ± 5 |

The apparent differences in binding of pyrrhocoricin to DnaK and GroEL suggests alterations in the binding mechanism or site of interaction. According to this assay, 4 μM unlabeled pyrrhocoricin competed for GroEL binding, and an increase in the peptide did not further modify the binding to the unlabeled analog. In contrast, the binding of the labeled peptide to DnaK decreased after preincubation with 4 μM pyrrhocoricin, and it could be further decreased upon increasing the amount of the competing unlabeled analog. This result may suggest that while GroEL has a single site for pyrrhocoricin binding, the interaction with DnaK involves two independent fragments of the protein.

Without wishing to be bound by theory, the inventors believe that the cationic antibacterial peptide family drosocin-pyrrhocoricin-apidaecin first faces the outer membrane of Gram-negative bacteria, and may destabilize it through binding to LPS. The peptides enter the outer membrane and encounter just a small resistance in the inner, bimolecular layer of the peptidoglycan. Upon internalization in the cells, they find DnaK in various bacterial compartments and deactivate it by strong binding. This theory explains the observations about the peptide family drosocin-pyrrhocoricinapidaecin: (a) The peptides are more active against Gram-negative strains than against Gram-positive strains. Gram-positive strains have a thicker peptidoglycan layer that is less permeable to the peptides; (b) The peptides kill E. coli D22 in lower concentrations than other E. coli strains. E. coli D22 has a permeable outer membrane, and no peptide is needed to destabilize it. The peptides freed from binding LPS are available for intracellular interaction with DnaK; (c) The peptides need 6-12 hours to kill bacteria. The first step, the internalization, is likely to proceed fast. However, the second step (i.e., deactivating DnaK to the level that results in bacterial death) can manifest only over a longer period of time; (d) All three peptides in the family enter the Gram-negative bacteria through binding to LPS; (e) All three peptides kill bacteria by inactivating DnaK, and (f) both the C- and the N-termini of the peptides are involved in binding to DnaK, or the efficacy differences between the two termini cannot be quantified by fluorescence polarization. According to this theory DnaK and GroEL are the transport proteins, and DnaK is also the final target. The competition fluorescence polarization assays suggest interaction of pyrrhocoricin at two independent sites with DnaK. The peptides may bind to DnaK weakly inside the conventional peptide binding pocket as well as strongly outside it. For the identification of a pyrrhocoricin-binding domain of DnaK outside the conventional peptide-binding pocket, the functional assay of Example 2 was performed to obtain an antibacterial profile of a broad spectrum pyrrhocoricin analog.

### EXAMPLE 2

### Strain Specificity of Antibacterial Activity of the Peptides

Growth inhibition assays are performed using the candidate antibacterial compounds and the Gram positive microorganisms Micrococcus luteus and Bacillus megaterium, and the Gram negative microorganisms, Escherichia coli D22, Agrobacterium tumefaciens, and Salmonella typhimurium. Antibacterial assays are performed in sterilized 96-well plates (Nunc F96 microtiter plates) with a final volume of 100 μl as described in Bulet (1996), cited above. Briefly, 90 μl of a suspension of a midlogarithmic phase bacterial culture at an initial 600 nm UV absorbance of 0.001 in Luria-Bertani rich nutrient medium is added to 10 μl of serially diluted candidate compounds in sterilized water. The final compound concentrations range between 0.15 and 80 μM, and more preferably between 0.3 μM and 40 μM. The plates are incubated at 30° C. for 24 hours with gentle shaking, and the growth inhibition is measured by recording the increase of the UV absorbance at 600 nm on an SLT Labinstruments 400 ATC microplate reader. The experiments are conducted over a 7-month period.

The inhibitory concentrations (IC₅₀) of each candidate compound is determined against each above-indicated microorganism. IC₅₀ is defined as the concentration in μM at that 50% growth inhibition of the selected microorganism is observed.

As one example, this in vitro antibacterial assay was performed on a broad spectrum divalent pyrrhocoricin analog, Chex-Pyrrhocoricin-2-19-Dap-[Chex-Pyrrhocoricin-2-19-Dap(Ac)], and the results illustrated in Table 4.

TABLE 4

| Microorganism | IC₅₀ in μM |
|---|---|
| Gram Negative Bacteria: | |
| E. coli D22 | 0.1-1.2 |
| S. typhimurium | 1.25-2.5 |
| P. aeruginosa | >40 |
| Erwinia carotovora carotovora | >40 |
| Gram Positive Bacteria: | |
| M. luteus | 40-80^{a} |
| B. megaterium | 2.5-5 |
| A. viridans | 1.2-5 |
| S. aureus | >40 |
| S. pyrogenes | >40 |

^{a}The assay was performed in poor broth medium, except for M. luteus which was done in Luria-Bertani rich nutrient medium.

According to this assay, the peptide killed E. coli, Salmonella typhimurium, Micrococcus luteus, Bacillus megeterium and Aerococcus viridans, but did not kill Pseudomonas aeruginosa, Erwinia carotovora carotovora, Staphylococcus aureus and Streptococcus pyogenes. The pyrrhocoricin analogs also kill Agrobacterium tumefaciens. The apparent lack of selectivity towards Gram-negative or Gram-positive strains further confirms that the killing of bacteria is not related strongly to membrane-binding. Rather, the specificity to certain bacterial strains may stem from altered binding to DnaK. In this case, at least one peptide-binding fragment should be sought in the variable domains of the protein. Careful comparison of various DnaK sequences reveal high homology N-terminal to the peptide-binding region, but considerably less homology downstream.

The structure of pyrrhocoricin makes it prone to bind both inside and outside the conventional peptide-binding region. Based on screening of DnaK-bound peptide libraries, DnaK recognizes extended peptide strands within and positively charged residues outside the substrate binding cavity. In perfect harmony, pyrrhocoricin displays a somewhat extended fragment in the middle of the sequence and positively charged residues all over, including the two bioactive termini [Otvos et al, Protein Science, cited above]. Peptide-binding at the C-terminal area of DnaK has been proposed at 518-545 residue stretch [J. Zhang and G. C. Walker, (1998) Arch. Biochem. Biophys., 356:177-186] that serves as a lid over the peptide-binding pocket. Another report [B. C. Freeman et al, (1995) EMBO J., 14:2281-2292] proposed that the highly negatively charged extreme C-terminal tetrapeptide of human Hsp70 binds a peptide substrate and affects ATP-ase activity. Yet another proof for C-terminal peptide binding comes from comparison of the inventor's peptide-blot with Western-blots developed with monoclonal antibodies directed against the C-terminal domain of mt Hsp70 [J. M. Green et al, (1995) Hybridoma, 14:347-354].

### EXAMPLE 3

### The Affinity of Antibacterial Proteins for Heat Shock Proteins

To characterize the affinity of various bacterial and mammalian heat shock proteins (as well as lipopolysaccharides originated from a large range of Gram-negative bacteria) for pyrrhocoricin, and for analog natural peptides such as drosocin, apidaecin and formaecin, the following steps are taken. The peptide-binding site(s) of DnaK are identified by using chemically synthesized fragments of the protein. The DnaK fragments are made individually by conventional chemical synthetic techniques. In an array format, the peptides are contacted with fluorescein- and biotin-labeled pyrrhocoricin and the amounts of pyrrhocoricin that bind the arrays, respectively, are measured by detection of the amount of label. To pinpoint potential peptide- or bacterial strain-dependent variations of the receptor, biotin-labeled peptide derivatives are used to isolate and characterize the target ‘receptor’ heat shock proteins from various Gram-positive and Gram-negative clinically relevant bacterial strains, such as various strains of Escherichia, Staphylococcus, Enterococcus, Pseudomonas and Gonorrhoeae. For Escherichia, the carboxy terminal of the DnaK protein is a target binding site.

### EXAMPLE 4

### Preparation of Pyrrocoricin Analogs

Pyrrhocoricin analogs are prepared for pharmaceutical or veterinary use. At low doses, pyrrhocoricin protected mice against E. coli infection, but, at higher doses was toxic to compromised animals. Analogs of pyrrhocoricin were therefore synthesized to further improve protease resistance and reduce toxicity. A number of such analogs are described in International Patent Publication No. WO 00/78956, published Dec. 28, 2000. These modified peptides are screened by the methods of this invention. Briefly, the above-referenced application provided a modified peptide that has antibacterial or anti-fungal activity, and has the formula [SEQ ID NO: 9]: R¹-Asp-Lys-Gly-X-Y-Leu-Pro-Arg-Pro-Thr-Pro-Pro-Arg-Pro-Ile-Tyr-X′-Y′-R² wherein R¹ is a moiety having a net positive charge;

wherein R² is selected from the group consisting of a free hydroxyl, an amide, an imide, a sugar and a sequence of one or up to about 15 additional amino acids, optionally substituted with a free hydroxyl, an amide, an imide or a sugar. These additional amino acids are independently selected from L-configuration or D-configuration amino acids. These additional amino acids are cyclized by the insertion of modifying sugars, imide groups and the like. These additional amino acids may also form spacers to cyclize the peptide by bridging between the N- and C-termini of the peptide;

wherein X and Y form a dipeptide that is Ser-Tyr or is a dipeptide formed of naturally occurring amino acids or unnatural amino acids, the dipeptide being resistant to cleavage by endopeptidases; and

wherein X′ and Y′ form a dipeptide that is Asn-Arg or is a dipeptide formed of naturally occurring amino acids or unnatural amino acids, the dipeptide being resistant to cleavage by endopeptidases. In one preferred embodiment, this peptide is a cyclic peptide in that R¹ and/or R² form an amino acid spacer (that is preferably a sequence duplicating at least a portion of the pyrrhocoricin peptide) linking the N- and C-terminal amino acids of the above formula. The peptides of this formula include modified peptides in which one or more conventional amide bonds between amino acids is replaced with a bond resistant to a protease, such as a thio-amide bond or a reduced amide bond. A linear derivative containing unnatural amino acids at the termini showed high potency and lack of toxicity in vivo. An expanded cyclic analog displayed broad activity spectrum in vitro.

A linear derivative containing unnatural amino acids at the termini showed high potency against E. coli infection and lack of toxicity in vivo and an expanded cyclic analog displayed broad activity spectrum in vitro.

The in vitro activity spectrums of these peptide derivatives are determined, followed by the required in vivo dosage and the toxicity. The in vitro testing is done on an E. coli model, as well as on clinically relevant bacterial strains, such as those listed in Example 3 above. The in vivo studies are conducted in mice with E. coli and Staphylococcus aureus as infective agents. Based upon the already characterized protease cleavage sites in mammalian sera, additional side-chain and backbone-modified analogs are synthesized and the in vitro and in vivo efficacy as well as the toxicity are assessed.

Among one of the useful peptides disclosed in the publication above and which binds to the D-E helix of the target sequence is the dimer [SEQ ID NO: 36].

### EXAMPLE 5

### In Vivo Antibacterial Activity Assay

An example of an in vivo antibacterial assay is performed as follows: Male mice of CD-1 strain (Harlan Sprague Dawley, Inc.) are intravenously infected in the tail with 1,000,000 colony forming units (0.2 ml) of a selected bacterium, e.g., Escherichia coli strain (ATCC Accession No. 25922). To obtain better infection, mice are also fed with the bacteria, in this case, E. coli. The candidate antibacterial compounds are intravenously injected 1 hour after infection at varying doses, e.g., 10, 25 and 50 mg/kg, followed by a booster injection after 5 hours of infection. Mice are observed at 1 hour, 5 hours, 1 day, and 2 days post-infection for clinical signs (e.g., decreased activity and head tilt) or mortality, and are compared with control mice who received 5% dextrose (DS5) instead of candidate compounds (negative control) or are submitted to the same candidate compound treatment, but received 50 mg/kg of DS5 instead of the bacteria (toxicity).

The mice are examined after several days for symptoms of infection, and the candidate compounds scored appropriately for antibiotic activity and stability.

An in vivo assay, identical to the last one (toxicity), is performed for studying the efficacy of the anti-mouse designed peptides or other molecules to kill mice. Two administration routes are used: feeding the mice or applying the peptides designed to terminate mice intravenously to identify possible advantageous delivery protocols.

For killing insects, the test molecules are added to the culture medium. If otherwise in vitro active peptides do not terminate the insects, the insects are hand-pricked with the molecules in the abdomen, similar to the assay described in Bulet et al., 1993, cited above).

### EXAMPLE 6

### Synthetic Peptides for Study of the Target Binding

The following DnaK fragments were synthesized:
a) E. coli DnaK aa397-439 of SEQ ID NO: 10, the conventional peptide binding pocket;
b) E. coli DnaK aa513-551 of SEQ ID NO: 10, the hinge region between C-terminal helices A and B, containing the entire B-helix, which is located just above the peptide binding pocket;
c) E. coli DnaK aa583-615 of SEQ ID NO: 10, the hinge region between C-terminal helices D and E, located also in the multihelical lid, slightly further away from the peptide binding pocket;
d) N-terminally truncated forms of the E. coli D-E helix peptide, such as aa588-615, aa590-615 and an N-terminally blocked aa591-615 analog of SEQ ID NO: 10;
e) S. aureus DnaK 554-585 [SEQ ID NO: 34], structural analog of the E. coli 583-615 peptide; and
f) E. coli DnaK aa596-637 of SEQ ID NO: 10, the flexible region between the multihelical lid and the extreme C-terminus.

The sequences of the native antibacterial peptides are as follows:

Drosocin GKPRPYSPRPTSHPRPIRV [SEQ ID NO: 1]

Pyrrhocoricin VDKGSYLPRPTPPRPIYNRN [SEQ ID NO: 3]

Apidaecin 1a GNNRPVYIPGPRPPHPRI [SEQ ID NO: 35]

Native drosocin and pyrrhocoricin are glycosylated on the underlined threonines, but as the attached sugar moieties are not required for the antibacterial activity, the peptides used in this study did not contain carbohydrate side-chains.

Other peptides included the negative control conantokin G; an N-methyl-D-aspartate (NMDA) receptor antagonist [Zhou, L.-M. et al, (1996) J. Neurochem., 66:620-628]; pyrrhocoricin made of all D-amino acids; magainin II, an antibacterial peptide that kills bacteria by disintegrating the membrane [Bechinger, B. et al, (1993) Protein Sci., 2: 2077-2084]; cecropin A, another membrane-active antimicrobial peptide [Steiner, H. et al, (1981) Nature, 292: 246-248]; buforin II, an antibacterial peptide that binds to bacterial DNA [Park, C. B. et al, (1998) Biochem. Biophys. Res. Commun, 244: 253-257]; Pyrr₁₋₉ and Pyrr₁₀₋₂₀ [aa109 and aa10-20 of SEQ ID NO: 3]; biotin-labeled L- and D-pyrrhocoricin; fluorescein-labeled pyrrhocoricin, drosocin and apidaecin [Otvos, L., Jr. et al, (2000) Biochemistry, 39:14150-14159]; Pyrr₁₋₉ and Pyrr₁₀₋₂₀ also labeled with fluorescein; fluorescein- and biotin-labeled—tubulin fragment aa434-445 serving as negative controls [Hoffmann, R. et al, (1997) J. Peptide Res., 50: 132-142; Otvos, L., Jr. et al, (1998) Protein and Peptide Lett., 5:207-213], and another negative control fluorescein-labeled peptide with the sequence NTDGSTDYGILQINSR [SEQ ID NO: 8].

Pyrrhocoricin, drosocin, apidaecin 1a, their fragments and labeled variants, conantokin G, the E. coli and S. aureus DnaK fragments as well as the negative control labeled peptides were made by standard solid-phase methods [Fields, G. B., and Noble, R. L. (1990) Int. J. Pept. Protein Res., 35: 161-214]. The peptides were purified by reversed-phase high performance liquid chromatography, and their integrity was verified by laser-desorption and electrospray ionization mass spectrometry. The actual peptide content of the lyophilized samples was chromatographically determined [Szendrei, G. I. et al, (1994) Eur. J. Biochem., 226: 917-924]. Buforin II was purchased from Sigma (St. Louis, Mich.), cecropin A and magainin 2 were purchased from Bachem (King of Prussia, Pa.).

### EXAMPLE 7

### Inhibition of ATPase Activity

The protein folding activity of the 70 kDa heat shock protein family is driven by their ATPase activity that regulates cycles of polypeptide binding and release [Liberek, K. et al, (1991) J. Biol. Chem.,266: 14491-14496]. Although the region responsible for ATPase actions have been identified at the amino-terminal half of the protein [Davis, J. E., et al, (1999) Proc. Natl. Acad. Sci., USA, 96:9269-9276], the ATPase activity is allosterically modulated by the C-terminal domain of human Hsp70 and its analog Hsc70 [Freeman, B. C. et al, (1995) EMBO J. 14: 2281-2292; Tsai, M.-Y., and Wang, C. (1994) J. Biol. Chem. 269:5958-5962].

To determine whether the proline-rich antibacterial peptides that were predicted to bind to DnaK between the peptide binding pocket and the C-terminus would interfere with ATPase activity, a recently developed continuous spectrophotometric (colorimetric) ATPase activity assay [Rieger, C. E. et al, (1997) Anal. Biochem. 246: 86-95] was used. This assay uses using 2-amino-6-mercapto-7-methylpurine ribonucleoside (MESG)/purine nucleoside phosphorylase reaction to detect the released inorganic phosphate (EnzChek ATPase determination kit from Molecular Probes, Eugene, USA). Assays were performed in 500 μL tubes in duplicates in a total volume of 125 μL containing 20 mM tris(hydroxymethyl)amino-ethane (Tris)-HCl, pH 7.6, 1 mM MgCl₂, 300 mM ATP (except in assaying the baseline), 5 μg of DnaK (recombinant DnaK protein from StressGen,Victoria, Canada) and 50 molar equivalents of the particular peptide, MESG and the purine nucleoside phosphorylase recommended by the manufacturer. After incubation at 22° C. for 30 min, 100 μL of the reaction mixture was transferred to a quartz cuvette and the ultraviolet (UV) absorbance at 360 nm was measured. The assay was run in a miniaturized form to increase the concentration and therefore the enzymatic activity of DnaK. In a larger, standard format the ATPase activity of DnaK without peptide addition was 6 pmol/μg/min, in line with the published data of 4 pmol/μg/min [Liberek, K. et al, (1991) Proc. Natl. Acad. Sci. USA 88: 2874-2878].

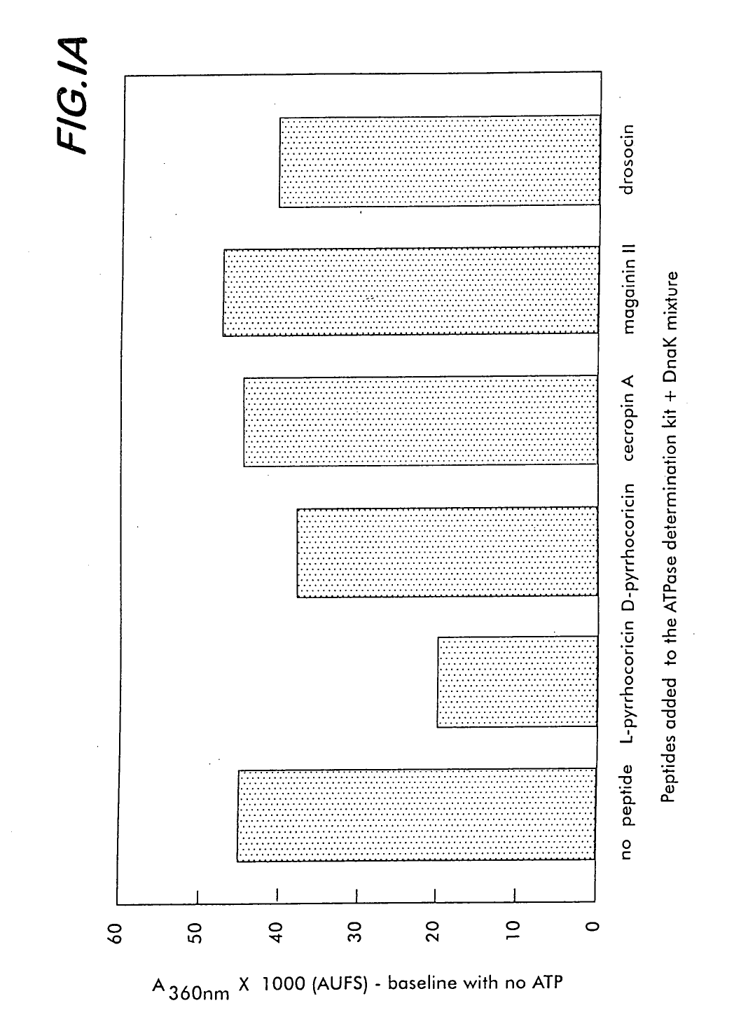

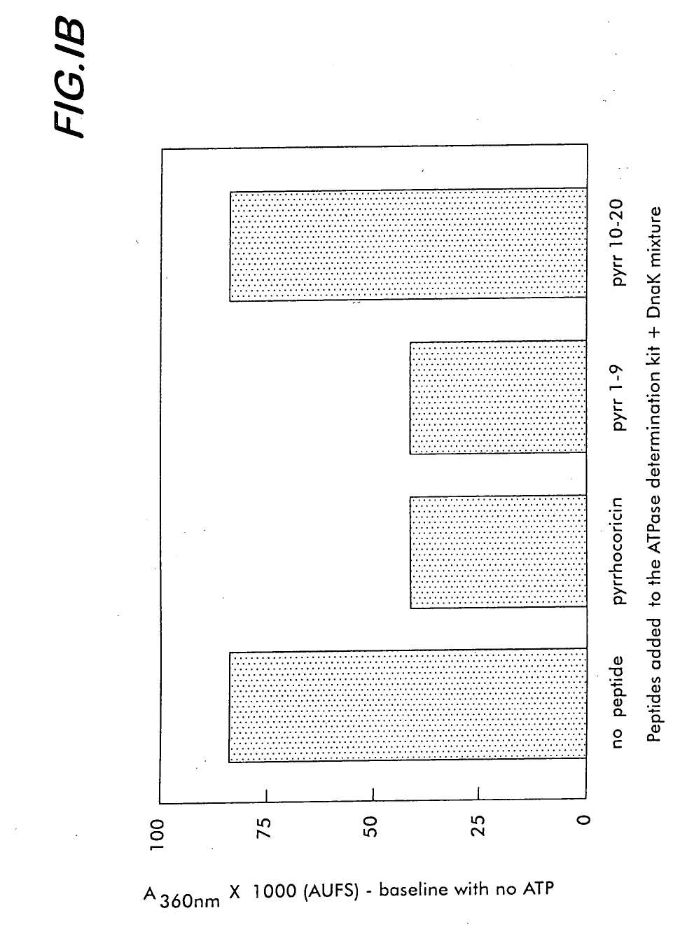

The inhibition of ATPase activity of recombinant E. coli DnaK by synthetic antibacterial peptides, L-pyrrhocoricin, D-pyrrhocoricin, cecropin A, magainin II and drosocin, in the EnzChek ATPase assay is shown in FIG. 1A; and the inhibition of ATPase activity of recombinant E. coli DnaK by synthetic pyrrhocoricin fragments, Pyrr_{AA1-9} and Pyrr_{AA10-20}, as well as the full length peptide in the EnzChek ATPase assay is shown in FIG. 1B.

Recombinant DnaK had a small, but measurable ATPase activity (FIG. 1A). The assay was repeated four times with different batches of DnaK, and freshly made reagent solutions. During these conditions, the increase of the UV absorbance at 360 nm upon addition of ATP varied from 0.038 to 0.077 AUFS, with a mean value of 0.060 AUFS, reflecting some differences in the quality of the various DnaK preparations. When the biologically active L-pyrrhocoricin was added to the assay mixture, the activity dropped to less than half of the original value (FIG. 1A). In contrast, the inactive D-analog of pyrrhocoricin had negligible effect. These assays were repeated twice and yielded the same reduction in the level of ATPase activity with the actual numbers dependent upon the original enzymatic activity of the different DnaK batches (compare with FIG. 1B).

Cecropin A, and magainin 2, two antimicrobial peptides that kill bacteria by disintegrating the membrane did not influence the ATPase activity of DnaK. Interestingly, drosocin, another proline-rich antibacterial peptide, a close relative of pyrrhocoricin, remained without affecting the ATPase activity (FIG. 1A). This suggested that pyrrhocoricin and drosocin did not share a common binding site to E. coli DnaK. Pyrrhocoricin did not influence the ATPase activity of recombinant Hsp70, the human equivalent of DnaK (0.058 vs. 0.063 AUFS).

Both termini of pyrrhocoricin are needed to kill bacteria, but the isolated halves alone, or their equimolar mixture, are completely inactive [Otvos, L., Jr. et al, (2000) Protein Sci. 9: 742-749]. To identify the fragment of pyrrhocoricin that is responsible for the inhibition of the ATPase activity in this assay, the results showed that when tested for the inhibition of the ATPase activity of recombinant DnaK, the amino terminal 1-9 fragment of pyrrhocoricin was as effective as full size pyrrhocoricin itself (FIG. 1B). The C-terminal 10-20 fragment also had some minor activity, but not as significant as the N-terminal half. Apparently, the amino-terminus is a strong binder to the allosteric ATPase site, but the C-terminal half also has some residues capable of binding to this DnaK domain.

### EXAMPLE 8

### Inhibition of Protein Folding as Assayed by Enzyme Activity of Live E. Coli Cultures

Current methods of measuring the protein folding efficiency of the heat shock proteins include measuring the catalytic potency of a number of enzymes produced by E. coli. Inhibition of the chaperone-assisted protein folding by the proline-rich peptides results in a decreased level of active enzyme production. This difference in the enzymatic activity can be detected. Alkaline phosphatase and β-galactosidase are two enzymes that are encoded by the E. coli TG-1 strain genome and abundantly expressed [Stec, B. et al, (2000) J. Mol. Biol. 299: 1303-1311; Nielsen, D. A. et al, (1983) Proc. Natl. Acad. Sci. USA 80: 5198-5202]. In fact, E. coli DnaK null mutants biosynthesize and secrete a number of enzymes at a significantly reduced level, including alkaline phosphatase and β-galactosidase [Wolska, K. I. et al, (2000) Microbios. 101: 157-168].

To reliably measure the enzymatic activity, bacteria in a colony number well exceeding that required by the standard antibacterial assay are required [Bulet, P. et al, (1996) Eur. J. Biochem. 238: 64-69]. A 5-mL culture was shaken at 37° C. for 5-6 hours, then 300 μL was added to 30 mL Luria-Bertani rich nutrient medium and the bacterial culture was shaken at 37° C. overnight. Sixteen μL of a 1 μg/μL peptide solution was added to 500 μL of the overnight culture and the mixture was incubated at 30° C. for 1-6 hours. After the incubation period expired, the cells were harvested with a 2-minute sonication on a probe sonicator and were centrifuged for 20 minutes at 3,000 g. The supernatant was used for the ensuing P-galactosidase and alkaline phosphatase assays.

A. P-Galactosidase Assay:

Fifty (50) μL of cell lysate was added into the wells of a 96-well plate. One hundred and ten μL of a 100 mM phosphate-buffered saline (PBS) pH 7.5 containing 1 mM MgSO₄/β-mercaptoethanol mixture (95:5, vol/vol) was added to the wells, the plate was covered and incubated at 37° C. for 5 minutes. Fifty μL of a 4 mg/mL ortho-nitrophenyl-β-D-galactopyranoside substrate solution was added to each well and the plate was incubated at 37° C. until the well contents turned bright yellow. The reaction was terminated by adding 90 μL of 1 M Na₂CO₃ solution and the plate was scanned by a microtiter dish reader set at 405 nm.

B. Alkaline Phosphatase Assay:

Fifty (50) μL of cell lysate was added into the wells of a 96-well plate. One hundred and ten μL of a 1.5 M 2-amino-2-methyl-1-propanol buffer, pH 10.3, was added to the wells, the plate was covered and incubated at 37° C. for 5 minutes. Fifty μL of a 4.9 mg/mL para-nitrophenyl disodium phosphate substrate solution was added to each well and the plate was incubated at 37° C. until the well contents turned bright yellow. The reaction was terminated by adding 90 μL of a 1 M H₃PO₄ solution and the plate was scanned by a microtiter dish reader set at 405 nm.

C. Results:

According to these assays, the peptides were added to the E. coli cultures at a concentration of 32 μg/mL (except L-pyrrhocoricin was added at either 32 μg/mL or 96 μg/mL as marked in the figures), which represents a value above the minimal inhibitory concentration of the active peptides, and is regarded as a conventional concentration for a series of standard antibacterial assays [Giacomenti, A. et al, (1999) Peptides 20: 1265-1273]. In this particular assay, the activities of either enzyme (without peptide addition) correspond to approximately 800 pmol/well/min. These experiments were repeated 2-3 times with bacteria growing in different rate as reflected by the increase of the enzymatic activity between 1 and 6 hours. FIGS. 2A and 2B show efficiently growing bacteria plated to duplicate (alkaline phosphatase) or single (β-galactosidase) wells.

Pyrrhocoricin strongly inhibited the β-galactosidase activity of an E. coli strain TG-1 culture in a peptide concentration-dependent manner (FIG. 2A). The inhibitory activity could be detected as early as 1 hour after introduction of the peptide. While it was not significantly inhibitory after I hour during this particular assay, drosocin became detrimental to the β-galactosidase activity after 6 hours. When the results of three independent assays were compared, drosocin inhibited the β-galactosidase activity in the entire 1-6 hour examination period (Table 5). None of the control peptides, including the all-D analog of pyrrhocoricin, the membrane-active peptide magainin 2, the DNA-binding antibacterial peptide buforin II, or the irrelevant peptide conantokin G had any β-galactosidase inhibitory effect on live E. coli cells (FIG. 2A and Table 5). Based on these results, pyrrhocoricin and drosocin inhibited chaperone-assisted protein folding. Both pyrrhocoricin and drosocin had a less dramatic effect on the alkaline phosphatase activity of the bacterial culture (FIG. 2B and Table 5). Nevertheless, the decreased enzymatic activity upon incubation with L-pyrrhocoricin and drosocin, compared with D-pyrrhocoricin, buforin II, magainin 2, or conantokin G is evident from FIGS. 2A and 2B. These tendencies were more visible when the experiment was repeated with less efficiently growing bacteria, although in this case the reading values were significantly lower and the experimental error became higher. Table 5 summarizes three independent assays for β-galactosidase and four assays for alkaline phosphatase inhibition. In spite of the sometimes observed high error rate, the table demonstrates well that only pyrrhocoricin and drosocin inhibit the activity of these enzymes in live E. coli cells.

Table 5 shows the results of three independent assays for β-galactosidase and four for alkaline phosphatase, run over a 3 week period. The high error value originated from the differences in the actual stage and rate of bacterial growth in the assay wells. Nevertheless, the data documents well that from all antibacterial peptides tested, only L-pyrrhocoricin and drosocin were inhibitory for the enzymatic activity of the bacterial cells. All peptides were applied at a final concentration of 32 μg/mL. The percentages were calculated based on the UV absorbance differences between the wells containing peptides relative to the wells containing distilled water and medium without cells. The above 100% values indicate UV absorbance below that for wells containing medium only; the negative values indicate UV absorbance above that for wells containing cells and distilled water.

TABLE 5

Inhibition of enzymatic
activity after 1 hour (%)

| Peptide | β-Galactosidase | Alkaline Phosphatase |
|---|---|---|
| L-pyrrhocoricin | 153 ± 57 | 32 ± 16 |
| D-pyrrhocoricin | (−4) ± 43 | (−6) ± 16 |
| Drosocin | 98 ± 68 | 35 ± 27 |
| Buforin II | (−5) ± 33 | (−6) ± 34 |
| Magainin 2 | (−50) ± 55 | (−23) ± 38 |
| Conantokin G | (−10) ± 10 | (−3) ± 23 |

In summary, although the peptides did not fully kill the larger batch of bacteria even if applied well over their minimal inhibitory concentration values, the changes in the enzymatic activity could be easily detected. Actually, the increase of enzymatic activity as the examination time progressed from 1 hour to 6 hours is useful as an internal control of the validity of the assay.

The success of connecting the antibacterial activity of pyrrhocoricin and drosocin with the mechanism of action as indicated by the β-galactosidase assay allows a reformulation of suitable assay conditions to gauge the efficacy of the proline-rich peptide family. For example, during these validated assay conditions pyrrhocoricin failed to kill even that particular E. coli strain (ATCC 25922) that had been used successfully for the in vivo efficacy assay described herein. These enzyme assays, especially the assay for the presence of β-galactosidase activity described herein, are suitable to assess the antibacterial efficacy of pyrrhocoricin-drosocin-apidaecin based peptides.

### EXAMPLE 9

### Identification of the Pyrrhocoricin-Binding Site on E. Coli DnaK

The inventors speculated that pyrrhocoricin binds to DnaK both inside and outside the conventional peptide binding pocket, and the most probable outside binding site is located between the peptide binding cavity and the extreme C-terminus. The allosteric inhibition of the ATPase activity, as presented above, supported this idea. This, together with the inhibition of the enzymatic activity of live bacteria, and therefore general inhibition of protein folding, suggested that the peptide bound somewhere in the region of the multihelical lid assembly.

To identify the actual pyrrhocoricin-binding site(s), four fragments of the protein were synthesized. These fragments corresponded to the peptide-binding cavity, the flexible C-terminus and two regions that included hinges between helices A and B as well as D and E. These C-terminal peptides were made because based on the biochemical data, the inventors hypothesized that pyrrhocoricin prevents protein folding by binding to one of these DnaK fragments, and permanently closes the lid over the peptide-binding pocket. Although an intrahelix hinge was also reported to operate in helix B [Mayer, M. P. et al, (2000) Nat. Struct. Biol. 7: 586-593], major movements of the multihelical lid likely involve the interhelix flexible domains.

To study the binding of biotin-labeled pyrrhocoricin to the DnaK fragments, the DnaK fragments were dissolved in electroblot transfer buffer (25 mM Tris, and 192 M glycine buffer containing 20% methanol), and were applied to a nitrocellulose membrane in 1 μg and 5 μg amounts. The membrane was blocked with 5% milk in a PBS-0.5% Tween 20 buffer (PBST) for 3 hours at room temperature and was incubated with 10 mL of 10 μg/mL biotin-labeled L-pyrrhocoricin, biotin-labeled D-pyrrhocoricin, and biotin-labeled tubulin 434-445 peptides dissolved in PBST containing 1% bovine serum albumin (BSA) for 1 hour. Biotin-labeled versions of the inactive D-pyrrhocoricin analog, and the unrelated peptide tubulin, were used as control peptides [Otvos, L., Jr. et al, (2000) Biochemistry, 39: 14150-14159]. After incubation, the membrane was extensively washed with PBST. Streptavidin conjugated to horseradish peroxidase (HRP) (Gibco-BRL) dissolved in 1% BSA-PBST was added to the membrane and was incubated at room temperature for 45 min. After extensive washing with PBST, the membrane was treated with chemiluminescence luminol-oxidizer (NEN) for 1 minute. The created chemiluminescence was exposed to a X-Omat blue XB-1 film (Kodak) for 10 seconds, and the film was developed. A control strip was stained with amido black 10B to verify the presence of all DnaK fragments on the nitrocellulose sheet.

Pyrrhocoricin and perhaps drosocin and apidaecin as well bind to DnaK at the multihelical lid region, located just above the conventional peptide-binding cavity. The function of this multihelical lid is the frequent opening and closing of the “entrance” to the pocket, and thereby regulating the protein folding process [Mayer, M. P. et al, (2000) Nat. Struct. Biol. 7: 586-593]. DnaK fragments that may constitute the binding site for the proline-rich antibacterial peptides can include those that form connections between the helices and can serve as a driving force for the opening and closing of the pocket. The most probable site was considered to be the hinge between helices A and B [Mayer, M. P. et al, (2000) Nat. Struct. Biol. 7: 586-593], although a latch around residues 536-538 of SEQ ID NO: 10, in the middle of helix B was also proposed to flip from a closed position in the adenosine 5′-diphosphate (ADP) state to an open position in the ATP state [Zhu, X. et al, (1996) Science 272: 1606-1014].

As demonstrated by the dot blot resulting from this example (not shown) a top row represented the blot developed with the effective antibacterial peptide L-pyrrhocoricin, a middle row represented the blot developed with the inactive D-pyrrhocoricin analog, and the bottom row represented the blot developed with tubulin. Earlier, a number of unspecific bands were detected on the Western-blot when the interaction between biotin-labeled peptides and the full-size DnaK protein had been studied. The non-specific binding was related to interaction with the peptide-binding pocket, as this DnaK fragment similarly bound all three (L-pyrrhocoricin, D-pyrrhocoricin, tubulin) peptides in this blot. Some unspecific binding was also observed to the C-terminal flexible domain.

In this dot blot, neither of the labeled antibacterial peptides bound to the DnaK aa513-551 fragment of SEQ ID NO: 10, that contains the potential movable domains of the hinge between A and B and the latch in helix B, referred to above. The fourth E. coli DnaK fragment, corresponding to the A-B helix region, was not stained. Remarkably, at 5 μg the bioactive L-pyrrhocoricin bound to another potential hinge region in DnaK, i.e., the hinge region at the junction between helices D and E, closer to helix E, at residues 590-615 of SEQ ID NO: 10. This binding site appeared to be specific, as only very weak staining was observed to biotin-labeled D-pyrrhocoricin or tubulin. This weak binding of drosocin to the D-E helix hinge fragment was approached from the D helix side.

The selectivity of pyrrhocoricin to some bacterial strains could be verified by the lack of binding to the E. coli aa583-615 of SEQ ID NO: 10 analog Staphylococcus aureus aa554-585 fragment [SEQ ID NO: 34]. Pyrrhocoricin and most of its designed analogs are inactive against S. aureus in vitro [Otvos, L., Jr. et al, (2000) Biochemistry, 39: 14150-14159].

The antimicrobial peptides produced by D. melanogaster and P. apterus kill bacteria by the same mechanism, but have slightly different binding sites on bacterial DnaK. This may reflect that different families of insects face different life-threatening bacteria. Alternatively, the peptides expressed by flies and bugs bind on shifted sites on DnaK to avoid a potential cross-reaction with the DnaK sequences of the individual insects themselves. However, this scenario would not explain the lack of drosocin binding to the E helix region of E. coli DnaK.

A comparison of the amino acid sequences of D. melanogaster DnaK 595-612 (ELTRHCSPIMTKMHQQGA) [SEQ ID NO: 11] and the corresponding E. coli DnaK 590-607 of SEQ ID NO: 10 (ELAQVSQKLMEIAQQQHA) reveals major dissimilarities, and suggests that a peptide capable of binding to the E. coli heat shock protein will not be able to attach to the insect's own DnaK.

### EXAMPLE 10

### Fluorescence Polarization

The binding of the synthetic DnaK fragments to their fluorescein-labeled pyrrhocoricin counterparts was also assessed in solution, by fluorescence polarization [Lundblad, J. R. et al, (1996) Mol. Endocrinol. 10: 607-612]. For these experiments, the unlabeled peptides were serially diluted in PBS (pH 7.4) or 0.1 M Tris-HCl (pH 8.0) containing 0.1 M ethylene-diamine-tetraacetic acid (EDTA) in 50 μL final volume in 6×50 mm disposable glass borosilicate tubes. The fluoresceinated peptides were added to each tube in a 50 μL aliquot to a final concentration of 1 nM and tubes were incubated at 37° C. for 5 minutes. The extent of fluorescence anisotropy was measured on a Beacon 2000 fluorescence polarization instrument (PanVera, Madison, Wis.) and calculated as millipolarization values. The filters used were 485 nm excitation and 535 nm emission with 3 nm band width. Non-linear curve fitting was done by using a dose-response logistical transition [y=a₀+a₁/(1+x/a₂)^{a}^{₃}] and the Levenberg-Marquardt Algorithm within the SlideWrite software package. The provided K_{d} value (a₂ coefficient) was calculated by the program.

A preliminary assay was run in PBS, in conditions and with controls identical to those used when pyrrhocoricin-binding of the full-size DnaK protein was studied [Otvos, L., Jr. et al, (2000) Biochemistry, 39: 14150-14159]. Due to the low solubility of the peptides, especially corresponding to the E. coli DnaK D-E helix 583-615 fragment of SEQ ID NO: 10, this peptide was replaced with a side-product of the synthesis. An N-terminally blocked analog of the aa591-615 fragment of SEQ ID NO: 10 exhibited somewhat increased solubility in PBS. The fluorescein-labeled pyrrhocoricin peptide bound strongly to the blocked E. coli DnaK fragment aa591-615 of SEQ ID NO: 10, and weakly to fragment aa397-439 of SEQ ID NO: 10, representing the conventional peptide binding pocket, verifying the results of the dot blot assay. No interaction above the level of the negative control conantokin G peptide was observed for the other two E. coli fragments, representing the A-B helix or the extreme C-terminus, or the D-E helix fragment of S. aureus DnaK. Fluorescein-labeled drosocin failed to bind to the blocked aa591-615 DnaK fragment of SEQ ID NO: 10.

As a reverse control the D-E helix hinge peptide was used against the fluorescein-labeled tubulin fragment. Again, no binding was detected. Nevertheless, due to the low solubility of the peptides, these data are presented here only in qualitative terms.

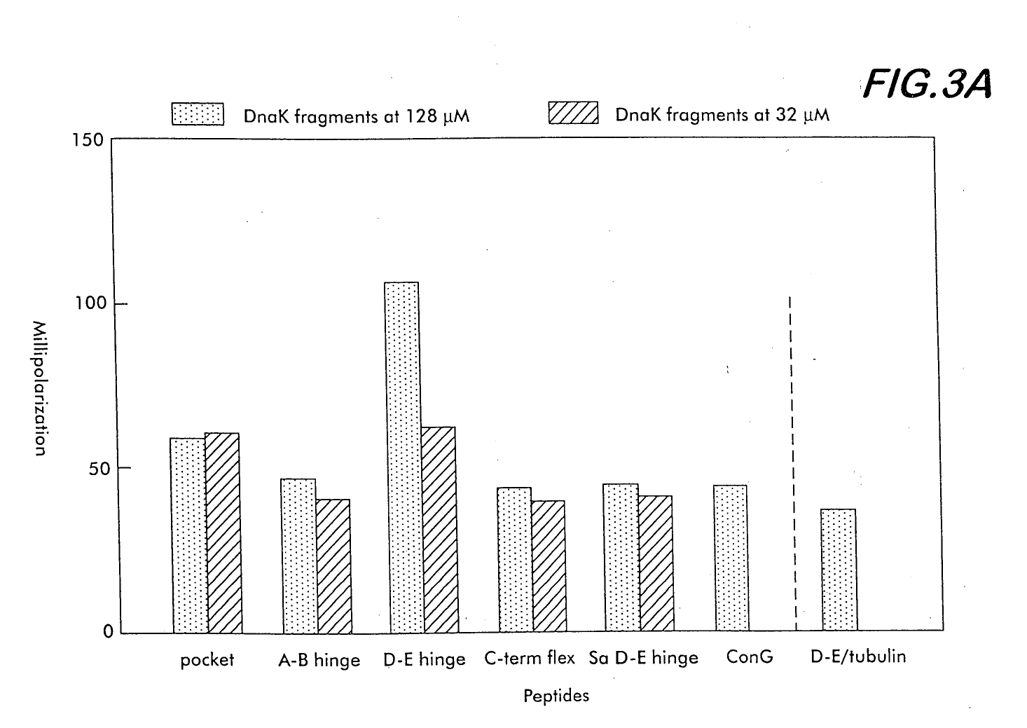

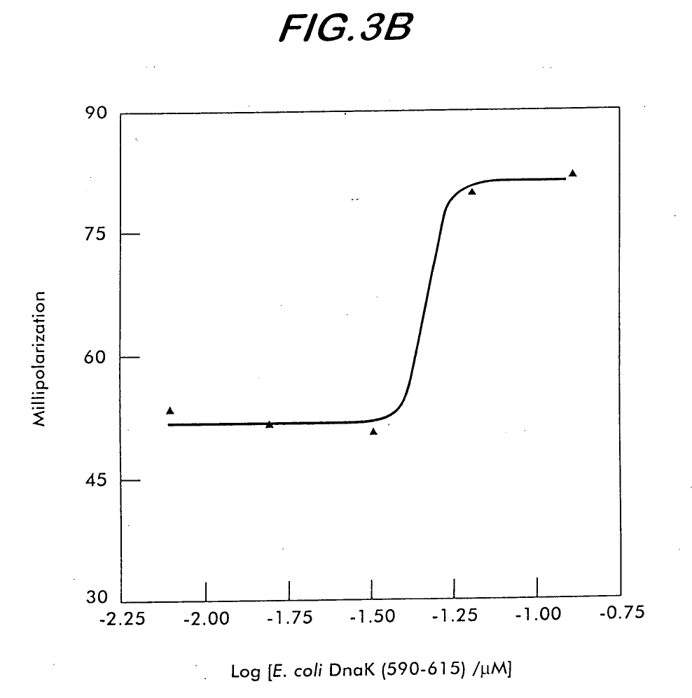

The assay was repeated in Tris HCl at pH 8.0, where the DnaK fragments exhibited increased solubility. DnaK fragments showing non-specific binding on the dot blot were not studied. The negative control fluorescein-labeled tubulin-peptide was replaced with another labeled peptide, which is not so heavily negatively charged, and potentially less cross-reactive. In Tris HCl, fluorescein-labeled pyrrhocoricin did not bind to the E. coli A-B helix fragment or the S. aureus D-E helix fragment over the labeled pyrrhocoricin background, which is indicated by the horizontal lines above the bars (FIG. 3A). Drosocin and apidaecin also failed to bind to the S. aureus D-E helix. In contrast, a concentration-dependent binding of pyrrhocoricin was observed to the E. coli D-E helix hinge region representing amino acids 583-615. This binding appears to be specific as the negative control fluorescein-labeled NTDGSTDYGILQINSR peptide [SEQ ID NO: 8] failed to bind to the same E. coli D-E helix (FIG. 3A). At a high concentration (128 μM) some binding to fluorescein-labeled drosocin and apidaecin was also observed.

To quantitatively characterize the pyrrhocoricin—D-E helix interaction, the complete binding curves were measured for E. coli DnaK fragments aa583-615 and aa588-615, both of SEQ ID NO: 10. The longer D-E helix peptide bound to the labeled pyrrhocoricin with a K_{d} of 50.8 μM (FIG. 3B). The shorter peptide bound with a somewhat decreased efficacy, exhibiting a K_{d} of 93 μM. This reduction in the binding affinity reflects either the decreased length of the second DnaK fragment or the inherent inaccuracy of the fluorescence polarization measurements.

Drosocin also bound to the aa588-615 fragment of SEQ ID NO: 10, but considerably weaker than pyrrhocoricin. This, together with the lack of drosocin binding to the blocked aa591-615 fragment of SEQ ID NO: 10 indicated that while pyrrhocoricin bound to the D-E helix region at the hinge and the E helix area, drosocin binding was somewhat shifted back to the N-terminal direction between the D helix and the hinge. This explains the differences in the ATPase activity inhibiting capacities between pyrrhocoricin and drosocin. When the DnaK binding of the fluorescein-labeled pyrrhocoricin halves were studied, both the Pyrr₁₋₉ and the Pyrr₁₀₋₂₀ fragments of SEQ ID NO: 3 strongly bound to the E. coli DnaK aa588-615 peptide of SEQ ID NO: 10 with a 30 millipolarization unit increase going from 32 μM to 160 μM, indicating that the binding to the DnaK fragment cannot be located only to the N-terminal, ATPase activity reducing segment.

Additional experiments to characterize the pyrrhocoricin-DnaK D-E helix interaction by isothermal titration calorimetry and surface plasmon resonance are currently underway, as is the identification of possible independent functions of this DnaK helix domain to establish the optimal conditions for later competitive binding studies.

### EXAMPLE 11

### Molecular Modeling

To have a well equilibrated structure for docking, the initial coordinate of pyrrhocoricin which was obtained from nuclear magnetic resonance spectroscopy (NMR) analyses [Otvos, L., Jr. et al, (2000) Protein Sci. 9: 742-749] was subjected to molecular dynamics (MD). Structures of the peptide were simulated in two 10 ns constant pressure and constant temperature MD in the presence of 5769 SPC/E water using the GROMACS 2.0 package [van der Spoel, D. et al, (1999) GROMACS User Manual version 2.0, Groningen, The Netherlands, http://md.chem.rug.nl/˜gmx]. Secondary structures of pyrrhocoricin in trajectories were determined by the DSSP method [Kabsch, W., and Sander, C. (1983) Biopolymers 22: 2577-2637].

The X-ray coordinate of E. coli, PDB ID: IDKX [Zhu, X. et al, (1996) Science 272: 1606-1014], was obtained from the Brookhaven Protein Data Bank [Berman, H. M. et al, (2000) Nucleic Acids Res. 28: 235-242]. The missing side chain atoms were reconstructed and all the missing H atoms were added with the SYBYL molecular modeling package. Since the C-terminal tail is missing from the X-ray structure, the protein was elongated with 9 residues in order to have the compatibility with the fluorescence polarization experiments. The structure of added sequence was set to a-helical and was energy minimized with the Tripos force field using the Kollman all charges and then the structure of the whole protein was energy minimized with the same parameters as above.

The structure of pyrrhocoricin was docked into DnaK using the FlexiDock module of SYBYL. The structure of DnaK was fixed in space, and side chains of residues 397 to 439 of SEQ ID NO: 10 (peptide-binding pocket) and residues 587 to 615 of SEQ ID NO: 10 (helices D and E) were flexible. All bonds, except the peptide bond, were set to be flexible in the structure of pyrrhocoricin. Genetic algorithm search was performed using 0.5 Å grid spacing, 60000 energy evaluation and saving the best 40 structures in a database.

Two, independently initiated, 10 ns simulations were performed to sample sufficiently the available conformational space for pyrrhocoricin. During both of the MD simulations, the total energy, temperature and density of the pyrrhocoricin peptide/solvent system came to equilibrium within the first 100 ps which time was excluded from the conformation analyses. In both simulations the following secondary structures were extensively sampled: -bridge conformations for residues 2 and 6; -turn conformations for residues 3 to 5 and residues 15 and 16. 0 verall, these data were in good agreement with the previous NMR measurement [Otvos, L., Jr. et al, (2000) Protein Sci. 9: 742-749]. Therefore as a characteristic structure, it was selected for flexible docking.

Two initial configurations were set up manually. Either the C-terminal part of pyrrhocoricin was placed into the peptide-binding pocket in such a way that the N-terminal domain of the peptide was close to the D helix region of the protein (docking 1) or the structure of pyrrhocoricin was aligned in antiparallel direction with the D-E helix region of E. coli DnaK (docking 2). During docking 1, pyrrhocoricin moved out from the peptide-binding pocket and became located in the area between the multihelical lid and the pocket. The modeling indicated that pyrrhocoricin did not preferably bind to the peptide-binding pocket (docking 1). The apparent conflict with the results of the dot blot and the fluorescence polarization could be resolved by considering that the physical measurements of the interaction did not provide the exact site of the binding. Actually, the peptide could have bound to an outer surface of the peptide-binding pocket, which is readily available in the synthetic, only partially folded protein fragment, but otherwise not accessible in full DnaK protein.

During docking 2, the orientation of pyrrhocoricin stayed antiparallel with helix E, and its N-terminal region stayed in close contact with the hinge and helix D. The conformation of the N-terminal region of pyrrhocoricin in the bound state resembled that of the isolated peptide, but from residue 14 a turn-like structure was stabilized which moved away the C-terminus of the peptide from helices D and E. The results of docking 2 are in full agreement with those of the fluorescence polarization measurements that showed that the N-terminal region of pyrrhocoricin (residues 1 to 9 of SEQ ID NO: 3) is the strongest binder to the D-E helix region of DnaK, and the binding surface probably extends further down to residues 11-12 of SEQ ID NO: 3. Apparently, the strong binding of pyrrhocoricin to the D-E helix hinge region permanently closes the lid over the peptide binding cavity, and prevents chaperone-assisted protein folding.

For modeling, the X-ray coordinates of DnaK of E. coli, PDB ID: I DKX [Zhu, X. et al, (1996) Science 272:1606-1014], are the only available known structures for heat shock proteins. For other bacterial and fungal heat shock proteins, as well as for Hsp proteins of other species having high sequence similarity to E coli DnaK, the three-dimensional structures are generated by homology modeling using the SWISS-MODEL [Peitsch, M. C. (1996) Biochem. Soc. Trans. 24: 274-279; Peitsch, M. C., and Guex, N. (1997) Large-scale comparative protein modeling. In: Proteome research: new frontiers in functional genomics, (Wilkins, M. R., Williams, K. L., Appel, R. O., and Hochstrasser, D. F., eds.) Springer. pp. 177-186] at the Expert Protein Analysis System proteomics server of the Swiss Institute of Bioinformatics (http://www.expasy.ch). Since all species heavily rely on functional DnaK, the sequence variations in the multihelical lid in general, and at the D-E helix junction in particular allow the design of peptides and peptidomimetics to control not only bacteria, but also fungi, mycobacteria, parasites, insects and rodents as well.

In the present docking techniques it is impossible to use implicit solvent molecules during docking, although it is well known that water molecules could substantially contribute to the stability of receptor-ligand complexes. To overcome this disadvantage, two independent procedures are used for docking. The FlexiDock module of SYBYL is used and the most characteristic peptide structures of molecular dynamics simulations are selected for docking.

Alternatively, the DOCK 4.0 program [Ewing, T. J. A. and Kuntz, I. D. (1997) J. Comp. Chem. 18: 1175-1189] is applied. In this latter method, both the ligand and the receptor will be rigid, therefore, to sample a large number of ligand conformations, every 10th structure from the molecular dynamics simulation will be docked. Although both docking procedures generate energy scoring giving the best ligand fitting, the relative stabilities of receptor-ligand complexes for two different ligand and/or receptor cannot be compared. Therefore, the binding free energy of the receptor ligand complexes is determined by using the chemical Monte Carlo/Molecular Dynamics [Massova, I., and Kollman, P. A. (1999) J. Am. Chem. Soc. 121: 8133-8143] and MM-PBSA [Eriksson, M., et al (1999) J. Med. Chem. 42: 868-881] modules of the AMBER 6 program package [Case, D. A. et al., (1999) AMBER version 6.0, University of California, San Francisco].

### EXAMPLE 12

### Method of Detecting HSP Inhibitors

The three dimensional atomic structures described above can be readily used as templates for selecting potent inhibitors. Various computer programs and databases, including those specifically identified above, are available for the purpose. A good inhibitor has at least excellent stearic and electrostatic complementarity to the target, a fair amount of hydrophobic surface buried and sufficient conformational rigidity to minimize entropy loss upon binding.

There are generally several steps in employing one of the above-described three dimensional structures as a template. First, a target region is defined. In defining a region to target, one can choose the active site cavity of the HSP, or any place that is essential to the protein refolding activity.

Second, a small molecule is docked onto the target using one of a variety of methods. Computer databases of three-dimensional structures are available for screening millions of small molecular compounds. A negative image of these compounds is calculated and used to match the shape of the target cavity. The profiles of hydrogen bond donor-acceptor and lipophilic points of these compounds are also used to complement those of the target. One skilled in the art can readily identify many small molecules or fragments as hits.

Third, one may link and extend recognition fragments. Using the hits identified by above procedure, one can incorporate different functional groups or small molecules into a single, larger molecule. The resulting molecule is likely to be more potent and have higher specificity than a single hit. It is also possible to try to improve the “seed” inhibitor by adding more atoms or fragments that will interact with the target protein. The originally defined target region can be readily expanded to allow further necessary extension.

A limited number of promising compounds is selected via this process. The compounds are synthesized and assayed for their inhibitory properties. The success rate is sometimes as high as 20%, and it may still be higher with the rapid progresses in computing methods.

### EXAMPLE 13

### Drug Design

The design of new drugs can be based on either mimicking the conformation of known ligands or on the structure of the peptide-binding domain of the receptor. The interaction of pyrrhocoricin, drosocin and apidaecin with the heat shock protein DnaK identifies DnaK as a convenient target for drug design. The bioactive conformation of the peptide-binding fragments of DnaK are observed for rational design of novel antibacterial drugs.

For example, the structure of native pyrrhocoricin and drosocin was determined by NMR and CD spectroscopy, and reverse-turns were identified as pharmacologically important elements at the termini, bridged by extended peptide domains. The ligand-binding fragment(s) of DnaK alone, and complexed with the strongest binding peptide ligands, are submitted to similar conformational analysis. The conformational analysis is facilitated by the available high resolution structure of DnaK and some of its ligand binding domains.

In another embodiment a synthetic molecule is designed to inhibit protein refolding activity of the heat shock protein (HSP). Such a compound has both high affinity and specificity for the HSP target sequence. Accordingly, a small molecule is designed that restricts the movement of helix D and E, thereby restricting the mobility of the hinge region therebetween and thus serve as a inhibitor of HSP function. Variations of these general strategies, such as modifying the peptide chemical nature and length, are also employed.

Small molecules are designed to bind one of these helices and thus disrupt protein folding activity. Since protein folding activity of HSP proteins requires some mobility of the helices, such compounds are useful in inhibiting HSP function.

### EXAMPLE 14

### Comparison of DNAK Target Sequences

The E. coli and other DnaK D-E helix sequences were compared in Table 6 below.

TABLE 6

| | | Identical/similar | |
|---|---|---|---|
| | | aa | SEQ |
| Organism | DnaK protein target sequence | scores | ID NO: |
| E. coli | I E A K M Q E L A Q V S Q K L M E I | | 6 |
| | A Q Q Q H A Q Q Q T A G A D A | | |
| S. typhiimurium | I E A K M Q E L A Q V S Q K L M E I | 30/3 | 26 |
| | A Q Q Q H A Q Q Q A G S A D A | | |
| A. tumefaciens | I Q A K T Q T L M E V S M K L G Q A | 9/11 | 15 |
| | I Y E A Q Q A E AG D A S A E | | |
| H. influenzae | I E A K I E A V I K A S E P L M Q A V | 9/9 | 16 |
| | Q A K A Q Q A G G E Q P Q Q | | |
| S. aureus | I K S K K E E L E K V I Q E L S A K V | 13/5 | 22 |
| | Y E Q A A Q Q Q Q Q A Q G A | | |
| S. pyogenes | M K A K L E A L N E K A Q A L A V K | 7/9 | 23 |
| | M Y E Q A A A A Q Q A A Q G A | | |
| C. albicans | Y E D K R K E L E S V A N P I I S G A | 6/9 | 24 |
| | Y G A A G G A P G G A G G F | | |
| human Hsp70 | F E H K R K E L E Q V C N P I I S G L | 7/8 | 27 |
| | Y Q G A G G P G P G G F G A | | |

From the 33 residues, the sequence alignments were observed and the identical/similar residue scores between the E. coli sequence and the others were scored (see col. 3). Over the first 24 residues of the above-described peptides, the scores were 24/0, 9/8, 8/9; 9/4; 7/8; 5/5 and 7/6, respectively.

The most concentrated area for amino acid mutations involve the hinge region extending to helix E: E. coli aa591-600 of SEQ ID NO: 10, AQVSQKLMEI.

According to this invention, a strain-specific antibacterial peptide can be designed by eliminating the flexibility between helices D and E and prevent opening and closing of the multihelical lid over the conventional peptide-binding pocket of DnaK.

Modeling is based on the published X-ray and NMR structure of E. coli DnaK, provided that the D-E helix region of the other, bacterial strain HSPs assume the same overall conformation. The known E. coli coordinates are used for homology modeling for other DnaK variants as well. The gross secondary structure of the various D-E helix peptides are then compared by circular dichroism spectroscopy (CD).

CD spectra are taken in water, and water-trifluoroethanol (TFE) mixtures to determine whether the characteristic unordered or turn→α-helix conformational transition, frequently observed for peptide fragments of helical domains of proteins, occurs at identical TFE concentrations. In pure water, the CD spectra of the E. coli and S. aureus DnaK fragments could be assigned as a type C spectrum, and reflect the dominance of type I (III) β-turns, or a mixture of type I and type II turns. Addition of 5% TFE (v/v) resulted in a redshift of both ππ* bands to 190 and 204 nm respectively, accompanied by an increase of the intensity of the positive band. This is an unmistakable indication of the appearance of helical structures, most likely 3₁₀-helices. The two DnaK fragments behaved identically, at least in spectral terms. While the spectral features of well-developed α-helices could already be seen at a TFE concentration as low as 10%, the intensity was increased with increasing TFE content. In 100% TFE a fully α-helical spectrum was recorded.

Significantly, the spectral features of the E. coli and S. aureus peptides remained very similar in all water-TFE compositions studied. If any difference could be detected it was a minor intensity increase throughout and some redshift (around 1 nm) at low TFE concentrations for the S. aureus sequence compared to the E. coli peptide. This can be explained by the increased number of potential salt bridges along the helix barrel. The S. aureus peptide contains 3 and 5 potential Glu-Lys salt bridges in i, i+3 and i, i+4 positions, respectively. These figures for the E. coli fragment are 2 and 0.

Taken together, the isolated peptide fragments exhibit all helical features of the complete DnaK multihelical lid, and they are very similar but not identical. This means that the amino acid alterations do result in minor structural changes, but the overall conformation being identical, the published X-ray and NMR coordinates of the D-E helix region of E. coli DnaK can be used as a basis of designing peptides capable of binding to the same fragment of other bacterial or fungal DnaK proteins.

To estimate the bound conformation of the antibacterial peptides, the spectra of drosocin or pyrrhocoricin alone, as well as those of the E. coli and S. aureus DnaK fragments were recorded, followed by the recording of the CD spectra of antibacterial peptide-DnaK fragment mixtures. Finally the original spectra of the DnaK peptides were subtracted from the spectra of the mixtures, and the residual spectra, representing the conformation of the bound peptides were compared to the spectra of the antibacterial peptides alone. This exercise is justifiable only if the conformation of the protein fragments remain unchanged upon interaction with drosocin or pyrrhocoricin. The antibacterial peptides demonstrate very low level of ordered secondary structure in 10% TFE, compared to the clearly helical DnaK fragments. It is expected that the binding will not modify the helix structure of the rigid DnaK fragments, but can influence the conformation of the flexible antibacterial peptides.

The following interactions were studied: pyrrhocoricin—E. coli DnaK aa583-615 of SEQ ID NO: 10, drosocin—E. coli DnaK aa583-615 of SEQ ID NO: 10, pyrrhocoricin—S. aureus aa554-585 [SEQ ID NO:34], and drosocin—S. aureus aa554-585 [SEQ ID NO:34]. The results of this conformational analysis for pyrrhocoricin—E. coli DnaK interaction are as follows. The CD spectrum of the mixture of the two peptides resembled that of the DnaK fragment alone, except that the intensities were lower, due to the lower intensity of the CD spectrum of pyrrhocoricin. When the spectrum of the DnaK peptide was subtracted from the spectrum of the mixture, a small, but unquestionably observable redshift of both pyrrhocoricin bands was detected, indicating that interaction with the heat shock protein fragment resulted in increasingly ordered structure of the antibacterial peptide.

To ascertain that this conformational change upon binding was not a spectroscopical artifact, the procedure was repeated with pyrrhocoricin and the DnaK peptide derived from the non-responsive strain S. aureus. In this case the wavelength of the pyrrhocoricin band maxima remained unchanged in the mixture, supporting the finding that pyrrhocoricin does not bind to the S. aureus DnaK D-E helix.

Finally, the interaction between drosocin and the E. coli DnaK peptide was studied. In contrast to pyrrhocoricin, DnaK binding did not appear to modify the conformation of drosocin. This finding is consistent with the earlier documented weaker binding of drosocin to the E. coli aa583-615 DnaK fragment of SEQ ID NO: 10, and may also reflect to the slightly N-terminally shifted binding site on E. coli DnaK of drosocin compared to pyrrhocoricin, which binds closer to the C-terminus.

Based on this information, it is not sufficient if the pyrrhocoricin analogs designed to possess increased resistance to serum proteases or improved pharmacokinetic properties show unchanged secondary structure compared to pyrrhocoricin alone. Such peptides and peptidomimetics to efficiently kill bacteria should resemble the bound conformation of pyrrhocoricin, the most active antibacterial peptide of this family known to date.

### EXAMPLE 15

### Peptide Design and Binding to the Synthetic HSP70 Fragments

The contact residues between pyrrhocoricin and the E. coli DnaK aa583-615 of SEQ ID NO: 10 fragment are identified by using multidimensional NMR techniques. After the contact residues between pyrrhocoricin or drosocin and the multihelical lid of DnaK are identified, pyrrhocoricin- and drosocin-based peptides and peptidomimetics are designed with computer methods to bind to the D-E helix hinge of the various Hsp70 sequences. The peptides are designed for selective binding to a given Hsp70 fragment, keeping in mind no or minimal cross-reaction with the other animal Hsp70 (DnaK) sequences and absolutely no binding to human Hsp70.

Table 6 compares the amino acid sequences of representative Hsp70 proteins starting from the beginning of helix D down to the end of helix E. These fragments are the strict homologs of the E. coli and S. aureus DnaK sequences used in Example 14, except that they end 6 residues earlier. This change was made because the homology modeling across the larger group of species reveals a large gap continuing further to the C-terminus and the sequences become non-comparable. The corresponding human Hsp70 sequence is used as a constant negative control ensure that the designed peptides are not toxic to humans. Other control DnaK fragments are those longer ones corresponding to E. coli and S. aureus.

TABLE 7

| | Amino Acid | | Sequence |
|---|---|---|---|
| Organisms | Sequence | Type | Helix D-hinge-helix E-flexible |
| Agrobacterium | 579-604 | Gram- | DDIQAK-TQT-LMEVSMKL- |
| tumefaciens | | negative | GQAIYEAQQ [SEQ ID NO: 28] |
| Streptococcus | 554-580 | Gram- | MKAKLEAL-NEKAQ-ALAVKM |
| pyogenes | | positive | YEQAAAAQ [SEQ ID NO: 29] |
| Saccharomyces | 579-615 | fungus | KEEFDDKLKEL-QDI-ANPIMSKL |
| cerevisiae | | (Baker's yeast) | YQAGG [SEQ ID NO: 30] |
| Plasmodium | 605-627 | parasite | LKQKLKDLEA VCQP IIVKL |
| falciparum | | (malaria) | YGQP [SEQ ID NO: 31] |
| Drosophila | 589-615 | insect (fruit | FDHKMEELTR HCSP IMTKMH |
| melanogaster | | fly) | QQGAGAA [SEQ ID NO: 32] |
| Mus musculus | 595-615 | rodent | YEHKQKELER VCNP IISKL YQ |
| | | (mouse) | [SEQ ID NO: 33] |
| Escherichia coli | 583-615 | Control | IEAKMQELA QVSQ KLMEIA |
| | | | QQQHAQQQTAGADA |
| | | | [aa 583-615 of SEQ ID NO: 10] |
| Staphylococcus | 554-585 | Control | IKSKKEELEK VIQ ELSAKVYE |
| aureus | | | QAAQQQQQAQG [SEQ ID NO: 34] |
| Homo sapiens | 592-624 | Control | FEHKRKELE QVCNP IISGL |
| | | | YQGAGGPGPGGFGA |
| | | | [SEQ ID NO: 27] |

As is apparent from Table 7, the sequences are remarkably different. Even the closest mouse—human pair has one conservative and three non-conservative amino acid alterations. These mutations seem to be sufficient to design peptides specific for the mouse sequence. Based on the model discussed above, pyrrhocoricin binds to the D-E helix region “backwards”, i.e. in an antiparallel fashion. It can interact with E. coli DnaK on three points. Arg9 and Arg19 anchor the antibacterial peptide to Glu599 of helix E and Glu590 of helix D in the heat shock protein DnaK. This orientation would overlay the Pro Arg Pro aal3-15 middle turn with the D-E helix hinge Val Ser Gln aa594-596 of SEQ ID NO: 10. The resulting three-point interaction prevents movements of the hinge. This theory explains pyrrhocoricin's inactivity against S. aureus, which lacks a negative charge in aa position 570 (it is an Ala; S. aureus residue 570 is the equivalent of E. coli Glu599). Based on this, Lys612 in the mouse protein can be used to anchor a negatively charged residue in the designed peptide. The human sequence lacks this potential positively charged anchor (it is a Gly).

In the models above, the structural motifs of E. coli DnaK are used to divide the protein fragments into the helix D-hinge-helix E-flexible categories. These categories may not perfectly fit the non-E. coli sequences. For example, the IISGL fragment of human DnaK more conceivably assumes a turn or 3₁₀-helix than an α-helix. However, in the context of the human Hsp70 protein, the IISGL fragment is still part of the multihelical lid assembly.

The designed peptides are chemically synthesized without any changes, with an N-terminally added biotin and with an N-terminally added fluorescein moiety. Standard Fmoc-chemistry is used throughout. Most of the amino acids are conventional L-residues. However, for a better fit to the DnaK sequences, and perhaps to stabilize the peptides against proteolytic attack, some natural residues will be replaced with non-natural amino acids. Incorporation of D-amino acids, frequently employed in peptide analog design appears to be unfavorable for biological activity of the pyrrhocoricins, and is omitted.

The charge, polarity or spatial requirements of given side-chains are maintained, or slightly modified if required, by incorporating various non-natural amino acids, from which appropriately Fmoc-protected derivatives are offered by a number of companies, including Neosystem Laboratoire (Strasbourg, France), RSP Amino Acid Analogues (Worcester, Mass.), Chem-Impex International (Wood Dale, Ill.) etc. These modified amino acid derivatives ready for peptide synthesis include single ring and polycyclic homoaromatic and heteroaromatic residues (Phe, Tyr, Pro, Trp mimics), amino-, alkylamino-, and guanidine containing side-chains (Lys, Arg), other heteroatom containing side-chains, other turn mimics, dipeptide units containing reduced amide bond between the residues, etc. A long range of amino acid diversity elements are marketed by Advanced Chemtech (Louisville, Ky.).

While the naked peptides are used for the biological studies, the labeled peptides are used for characterizing the binding properties to the DnaK fragments. The solid-phase binding is studied with biotin-labeled peptides and dot blot, and the solution binding (including the binding constant) is determined with the fluorescein-labeled peptides and fluorescence polarization techniques as described in the preceding examples.

All documents cited above are incorporated by reference herein, including the provisional priority U.S. patent application Nos. 60/177,565 and 60/237,599. This invention is not to be limited in scope by the specific embodiments described herein. Indeed, various modifications of the invention in addition to those described herein will become apparent to those skilled in the art from the foregoing description. Such modifications are intended to fall within the scope of the appended claims. The disclosures of the patents, patent applications and publications cited herein are incorporated by reference in their entireties.

1. A method for identifying a compound that has a biocidal effect against a selected non-human organism, said method comprising screening from among known or unknown molecules, a test molecule that binds selectively to a target sequence of a multi-helical lid of a heat shock protein of said selected non-human organism, wherein said binding inhibits the protein folding activity of said protein.

2. The method according to claim 1, wherein said protein comprises multiple hinge regions flanked by adjacent helices, and wherein said binding physically restrains essential movement of at least one hinge region.

3. The method according to claim 1, wherein said binding is covalent or non-covalent.

4. The method according to claim 1, wherein said molecule does not bind or restrain the movement of a heat shock protein of a mammal which is exposed to said molecule.

5. The method according to claim 1, wherein said target sequence is at least 65% homologous to the E. coli DnaK D-E helix domain of the sequence IEAKMQELAQVSQKLMEIAQQQHAQQQTAGADA SEQ ID NO: 6, or to a fragment thereof.

6. The method according to claim 1, wherein said target sequence is at least 65% homologous to D-E helix domains selected from the group consisting of
(a) IEAKMQELAQVSQKLMEIAQQQHAQQQ AGSADA SEQ ID NO:26;
(b) IQAKTQTLMEVSMKLGQAIYEAQQAEAG DASAE SEQ ID NO:15;
(c) IEAKIEAVIKASEPLMQAVQAKAQQAGG EQPQQ SEQ ID NO: 16;
(d) IKSKKEELEKVIQELSAKVYEQAAQQQQ QAQGA SEQ ID NO: 22;
(e) MKAKLEALNEKAQALAVKMYEQAAAA QQAAQGA SEQ ID NO:26;
(f) YEDKRKELESVANPIISGAYGAAGGAPG GAGGF SEQ ID NO: 24; and
(g) a fragment of any one of (a) through (f).

7. The method according to claim 6, wherein said fragment comprises residues 1-24 of (a) through (f).

8. The method according to claim 6, wherein said homologous sequences differ at one or more amino acid residues of SEQ ID NO: 6 selected from the group consisting of: E2, M5, E7, A9, Q20, Q13, and M16.

9. A composition comprising:
(a) a synthetic, non-naturally occurring molecule that binds to a selected multi-helical lid of a heat shock protein of a selected organism, wherein said molecule inhibits the protein folding activity of said protein, and
(b) a suitable carrier,
whereby exposure of said organism to said composition retards the growth and reproduction thereof.

10. The composition according to claim 9, wherein said heat shock protein comprises multiple hinge regions flanked by adjacent helices, and wherein said binding physically restrains essential movement of at least one hinge region.

11. The composition according to claim 9, wherein said organism is selected from the group consisting of a bacterium, a fungus, a parasite, a mycobacterium, an insect, and an animal.

12. The composition according to claim 9, wherein said molecule binds to a target sequence at least 65% homologous to at least one of the sequences selected from the group consisting of:
(a) IEAKMQELAQVSQKLMEIAQQQHAQQQ TAGADA SEQ ID NO:6;
(b) IEAKMQELAQVSQKLMEIAQQQHAQQQ AGSADA SEQ ID NO:26;
(c) IQAKTQTLMEVSMKLGQAIYEAQQAEAG DASAE SEQ ID NO:15;
(d) IEAKIEAVIKASEPLMQAVQAKAQQAGG EQPQQ SEQIDNO: 16;
(e) IKSKKEELEKVIQELSAKVYEQAAQQQQ QAQGA SEQIDNO:22;
(f) MKAKLEALNEKAQALAVKMYEQAAAA QQAAQGA SEQIDNO:26;
(g) YEDKRKELESVANPIISGAYGAAGGAPG GAGGF SEQ ID NO: 24; and
(h) a fragment of any one of (a) through (g).

13. The composition according to claim 12, wherein said fragment comprises residues 1-24 of any of sequence (a) through (g).

14. The composition according to claim 12, wherein said sequence differs at one or more amino acid residues of SEQ ID NO: 6 selected from the group consisting of: E2, M5, E7, A9, Q10, Q13, and M16.

15. A method of treating a mammal for a pathogenic infection comprising administering to said mammal a composition of claim 9.

16. A method of eliminating a plant, insect or animal pest comprising administering to a site of said pest a composition of claim 9.

17. A method for designing a compound that has a biocidal effect against a selected organism, said method comprising the step of:
modifying or synthesizing a molecule to bind selectively to, and physically restrain the essential movement of, a target sequence of a heat shock protein of said selected organism, wherein said compound inhibits the protein folding activity of said protein.

18. The method according to claim 17, wherein said compound binds to a sequence of said protein that is at least 64% homologous to a sequence selected from the group consisting of
(a) IEAKMQELAQVSQKLMEIAQQQHAQQQ TAGADA SEQ ID NO: 6;
(b) IEAKMQELAQVSQKLMEIAQQQHAQQQ AGSADA SEQ ID NO:26;
(c) IQAKTQTLMEVSMKLGQAIYEAQQAEAG DASAE SEQ ID NO:15;
(d) IEAKIEAVIKASEPLMQAVQAKAQQAGG EQPQQ SEQ ID NO:16;
(e) IKSKKEELEKVIQELSAKVYEQAAQQQQ QAQGA SEQ ID NO:22;
(f) MKAKLEALNEKAQALAVKMYEQAAAA QQAAQGA SEQ ID NO:26;
(g) YEDKRKELESVANPIISGAYGAAGGAPG GAGGF SEQ ID NO:24; and
(h) a fragment of any one of (a) through (g).

19. The method according to claim 18, wherein said fragment comprises residues 1-24 of (a) through (g).

20. The method according to claim 18, wherein said homologous sequences differ at one or more amino acid residues of SEQ ID NO:6 selected from the group consisting of: E2, M5, E7, A9, Q10, Q13, and M16.


SEQUENCE LISTING (36 sequences)

SEQ ID NO: 1 - protein, 19 aa, Drosophila melanogaster
GKPRPYSPRPTSHPRPIRV

SEQ ID NO: 2 - protein, 16 aa, Myrmecia gulosa
GRPNPVNNKPTPYPHL

SEQ ID NO: 3 - protein, 20 aa, P. apterus
VDKGSYLPRPTPPRPIYNRN

SEQ ID NO: 4 - protein, 18 aa, Apis mellifera
GNNRPVYIPQPRPPHPRI

SEQ ID NO: 5 - protein, 18 aa, Phormia terranovae
DEKPKLILPTPAPPNLPQ

SEQ ID NO: 6 - protein, 33 aa, E. coli
IEAKMQELAQVSQKLMEIAQQQHAQQQTAGADA

SEQ ID NO: 7 - protein, 20 aa, insect antibacterial peptide
VDKGRYLEAPTRPRPERNRK

SEQ ID NO: 8 - protein, 16 aa, negative control fluorescein-labeled peptide
NTDGSTDYGILQINSR

SEQ ID NO: 9 - protein, 18 aa, modified pyrrhocoricin peptide
  misc_feature (1)..(1): A moiety having a net positive charge is attached to Asp
DKGXXLPRPTPPRPIYXX

SEQ ID NO: 10 - protein, 638 aa, Escherichia coli
MGKIIGIDLGTTNSCVAIMDGTTPRVLENAEGDRTTPSIIAYTQDGETLVGQPAKRQAVTNPQNTLFAIKRLIGRRFQDEEVQRDVSIMPFKIIAADNGDAWVEVKGQKMAPPQISAEVLKKMKKTAEDYLGEPVTEAVITVPAYFNDAQRQATKDAGRIAGLEVKRIINEPTAAALAYGLDKGTGNRTIAVYDLGGGTFDISIIEIDEVDGEKTFEVLATNGDTHLGGEDFDSRLINYLVEEFKKDQGIDLRNDPLAMQRLKEAAEKAKIELSSAQQTDVNLPYITADATGPKHMNIKVTRAKLESLVEDLVNRSIEPLKVALQDAGLSVSDIDDVILVGGQTRMPMVQKKVAEFFGKEPRKDVNPDEAVAIGAAVQGGVLTGDVKDVLLLDVTPLSLGIETMGGVMTTLIAKNTTIPTKHSQVFSTAEDNQSAVTIHVLQGERKRAADNKSLGQFNLDGINPAPRGMPQIEVTFDIDADGILHVSAKDKNSGKEQKITIKASSGLNEDEIQKMVRDAEANAEADRKFEELVQTRNQGDHLLHSTRKQVEEAGDKLPADDKTAIESALTALETALKGEDKAAIEAKMQELAQVSQKLMEIAQQQHAQQQTAGADASANNAKDDDVVDAEFEEVKDKK

SEQ ID NO: 11 - protein, 18 aa, Drosophila melanogaster
ELTRHCSPIMTKMHQQGA

SEQ ID NO: 12 - protein, 21 aa, biotin-K-pyrrhocoricin
  misc_feature (1)..(1): biotin is attached to Lys in position 1
KVDKGSYLPRPTPPRPIYNRN

SEQ ID NO: 13 - protein, 18 aa, E. coli
DTTTIIDGVGEEAAIQGR

SEQ ID NO: 14 - protein, 16 aa, E. coli
FINKPETGAVELESPF

SEQ ID NO: 15 - protein, 33 aa, A. tumefaciens
IQAKTQTLMEVSMKLGQAIYEAQQAEAGDASAE

SEQ ID NO: 16 - protein, 33 aa, H. influenzae
IEAKIEAVIKASEPLMQAVQAKAQQAGGEQPQQ

SEQ ID NO: 17 - protein, 6 aa, E. coli
  misc_feature (5)..(5): amino acid can be Leu or Ile
SVSDXD

SEQ ID NO: 18 - protein, 6 aa, E. coli
  misc_feature (2)..(2): amino acid can be Ile or Leu
TXXDGV

SEQ ID NO: 19 - protein, 4 aa, E. coli
  misc_feature (2)..(2): amino acid can be Leu or Ile
EXES

SEQ ID NO: 20 - protein, 6 aa, E. coli
  misc_feature (4)..(4): amino acid can be Leu or Ile
FNLXDG

SEQ ID NO: 21 - protein, 18 aa, Apis mellifera
GNNRPVYIPGPRPPHPRI

SEQ ID NO: 22 - protein, 33 aa, S. aureus
IKSKKEELEKVIQELSAKVYEQAAQQQQQAQGA

SEQ ID NO: 23 - protein, 33 aa, S. pyogenes
MKAKLEALNEKAQALAVKMYEQAAAAQQAAQGA

SEQ ID NO: 24 - protein, 33 aa, C. albicans
YEDKRKELESVANPIISGAYGAAGGAPGGAGGF

SEQ ID NO: 25 - protein, 21 aa, fluorescein-K pyrrhocoricin
  misc_feature (1)..(1): fluorescein is attached to Lys in position 1
KVDKGSYLPRPTPPRPIYNRN

SEQ ID NO: 26 - protein, 33 aa, S. typhiimurium
IEAKMQELAQVSQKLMEIAQQQHAQQQAGSADA

SEQ ID NO: 27 - protein, 33 aa, Human Hsp70
FEHKRKELEQVCNPIISGLYQGAGGPGPGGFGA

SEQ ID NO: 28 - protein, 26 aa, Agrobacterium tumefaciens
DDIQAKTQTLMEVSMKLGQAIYEAQQ

SEQ ID NO: 29 - protein, 27 aa, Streptococcus pyogenes
MKAKLEALNEKAQALAVKMYEQAAAAQ

SEQ ID NO: 30 - protein, 27 aa, Saccharomyces cerevisiae
KEEFDDKLKELQDIANPIMSKLYQAGG

SEQ ID NO: 31 - protein, 23 aa, Plasmodium falciparum
LKQKLKDLEAVCQPIIVKLYGQP

SEQ ID NO: 32 - protein, 27 aa, Drosophila melanogaster
FDHKMEELTRHCSPIMTKMHQQGAGAA

SEQ ID NO: 33 - protein, 21 aa, Mus musculus
YEHKQKELERVCNPIISKLYQ

SEQ ID NO: 34 - protein, 32 aa, Staphylococcus aureus
IKSKKEELEKVIQELSAKVYEQAAQQQQQAQG

SEQ ID NO: 35 - protein, 18 aa, Apidaecin la
GNNRPVYIPGPRPPHPRI

SEQ ID NO: 36 - protein, 18 aa, modification of Pyrrhocoricin
  misc_feature (1)..(1): Asp in position 1 is modified by a 1-aminocyclo-hexane carboxylic
DLGSYLPRPTPPRPIYNR